(19) United States
(12) Patent Application Publication
(10) Pub. No.: US 2010/0223691 A1
(43) Pub. Date: Sep. 2, 2010

(54) TARGETED NUCLEOTIDE EXCHANGE WITH IMPROVED MODIFIED OLIGONUCLEOTIDES

(75) Inventor: Paul Bundock, Amsterdam (NL)

Correspondence Address: FOLEY AND LARDNER LLP;SUITE 500, 3000 K STREET NW, WASHINGTON, DC, 20007, US

(73) Assignee: KEYGENE N.V.

(21) Appl. No.: 12/666,154

(22) PCT Filed: Jun. 19, 2008
(86) PCT No.: PCT/NL08/00151

§ 371 Date: Jan. 29, 2010

(30) Foreign Application Priority Data
Jun. 22, 2007 (NL) PCT/NL2007/000159

(51) Int. Cl. A01H 1/00; C07H 21/04; C12P 19/34; C12N 5/10; C12N 1/19; C12N 1/15

(52) U.S. Cl. 800/278; 536/23.1; 435/91.1; 435/352; 435/419; 435/254.11; 435/363; 435/366; 435/254.2

(57) ABSTRACT

A method and oligonucleotides for targeted nucleotide exchange of a duplex DNA sequence, wherein the donor oligonucleotide contains at least two modified nucleotides, at least one of which is a propynylated purine and/or pyrimidine and at least one of which is a LNA having a higher binding affinity compared to naturally occurring A, C, T or G and/or binds stronger to a nucleotide in an opposite position in the first DNA sequence as compared to a naturally occurring nucleotide complementary to the nucleotide in the opposite position in the first DNA sequence.

### FIELD OF THE INVENTION

The present invention relates to a method for the specific and selective alteration of a nucleotide sequence at a specific site of the DNA in a target cell by the introduction into that cell of an oligonucleotide. The result is the targeted alteration of one or more nucleotides so that the sequence of the target DNA is converted where they are different. More in particular, the invention relates to the targeted nucleotide exchange using modified oligonucleotides. The invention further relates to oligonucleotides and kits. The invention also relates to the application of the method.

### BACKGROUND OF THE INVENTION

Genetic modification is the process of deliberately creating changes in the genetic material of living cells with the purpose of modifying one or more genetically encoded biological properties of that cell, or of the organism of which the cell forms part or into which it can regenerate. These changes can take the form of deletion of parts of the genetic material, addition of exogenous genetic material, or changes in the existing nucleotide sequence of the genetic material. Methods for the genetic modification of eukaryotic organisms have been known for over 20 years, and have found widespread application in plant, human and animal cells and micro-organisms for improvements in the fields of agriculture, human health, food quality and environmental protection. The common methods of genetic modification consist of adding exogenous DNA fragments to the genome of a cell, which will then confer a new property to that cell or its organism over and above the properties encoded by already existing genes (including applications in which the expression of existing genes will thereby be suppressed). Although many such examples are effective in obtaining the desired properties, these methods are nevertheless not very precise, because there is no control over the genomic positions in which the exogenous DNA fragments are inserted (and hence over the ultimate levels of expression), and because the desired effect will have to manifest itself over the natural properties encoded by the original and well-balanced genome. On the contrary, methods of genetic modification that will result in the addition, deletion or conversion of nucleotides in predefined genomic loci will allow the precise modification of existing genes.

Oligonucleotide-directed Targeted Nucleotide Exchange (TNE) is a method that is based on the delivery into the eukaryotic cell nucleus of synthetic oligonucleotides (molecules consisting of short stretches of nucleotide-like moieties that resemble DNA in their Watson-Crick basepairing properties, but may be chemically different from DNA) (Alexeev and Yoon, Nature Biotechnol. 16: 1343, 1998; Rice, Nature Biotechnol. 19: 321, 2001; Kmiec, J. Clin. Invest. 112: 632, 2003). By deliberately designing a mismatch nucleotide in the homology sequence of the oligonucleotide, the mismatch nucleotide may induce changes in the genomic DNA sequence. This method allows the conversion of single or at most a few nucleotides in the target, but may be applied to create stop codons in existing genes, resulting in a disruption of their function, or to create codon changes, resulting in genes encoding proteins with altered amino acid composition (protein engineering).

Targeted nucleotide exchange (TNE) has been described in plant, animal and yeast cells. The first TNE reports utilized a so-called DNA:RNA self-complementary oligonucleotide that is designed to intercalate at the chromosomal target site. The chimera contains a mismatched nucleotide on the DNA strand that forms the template for introducing the mutation at the chromosomal target. The first examples of TNE using chimeric DNA:RNA oligonucleotides came from animal cells (reviewed in Igoucheva et al. 2001 Gene Therapy 8, 391-399). Extensive research by many laboratories has shown that the TNE frequency using such oligonucleotides is variable, and on average very low, and depends on such factors as the transcriptional status of the target, the influence of the cell cycle and cell type transformed. TNE using chimeric DNA:RNA oligonucleotides has also been demonstrated in plant cells (Beetham et al. 1999 Proc. Natl. Acad. Sci. USA 96: 8774-8778; Zhu et al. 1999 Proc. Natl. Acad. Sci. USA 96, 8768-8773; Zhu et al. 2000 Nature Biotech. 18, 555-558; Kochevenko et al. 2003 Plant Phys. 132: 174-184; Okuzaki et al. 2004 Plant Cell Rep. 22: 509-512). In general, the frequencies reported in both plant and animal studies were too low for practical application of TNE on non-selectable chromosomal loci.

TNE using chimeric oligonucleotides was also found to be difficult to reproduce (Ruiter et al. (2003) Plant Mol. Biol. 53, 715-729), resulting in a search for alternative oligonucleotide designs giving more reliable results. Several laboratories have focussed on the use of single stranded (ss) oligonucleotides for TNE. These have been found to give more reproducible results in both plant and animal cells (Liu et al. 2002 Nuc. Acids Res. 30: 2742-2750; review, Parekh-Olmedo et al. 2005 Gene Therapy 12: 639-646; Dong et al. 2006 Plant Cell Rep. 25: 457-65).

Several groups have shown that the process of TNE can be mimicked in vitro using total cellular protein extracts. Such assays for TNE activity are called cell free assays.

The assay involves the repair of a mutation that inactivates a bacterial reporter gene (such as LacZ or an antibiotic resistance gene) by incubation of a plasmid carrying such a reporter together with an oligonucleotide and cellular protein from a particular cell type. After incubation, the plasmid is electroporated into E. coli which is used as a readout system to determine the TNE efficiency. The cell free system has been used in combination with both chimeric DNA:RNA (Cole-Strauss et al. 1999 Nucleic Acids Res. 27: 1323-1330; Gamper et al. 2000 Nucleic Acids Res. 28, 4332-4339; Kmiec et al. 2001 Plant J. 27: 267-274; Rice et al. 2001 40: 857-868; Thorpe et al. 2002 J. Gene Medicine 4, 195-204) and single stranded oligonucleotides (Igoucheva et al. 2001 Gene Therapy 8, 391-399; Kren et al. 2003 DNA Repair 2, 531-546; Olsen et al. 2005 J. Gene Medicine 7, 1534-1544).

In such experiments, the oligonucleotide effects a substitution, usually by changing a stop codon (TAG) into a codon specifying an amino acid. Furthermore, the cell free system can also be used to study the possibility of using oligonucleotides to produce single nucleotide insertions. Plasmids can be produced which have a single nucleotide deleted from the bacterial reporter gene, generating a frame shift mutation. In the cell free assay, the deletion is repaired by addition of the deleted nucleotide mediated by the oligonucleotide. Similarly, oligonucleotides containing one or more extra nucleotide not originally present in the target can also be used to introduce one or more nucleotides into the target sequence.

The greatest problem facing the application of TNE in cells of higher organisms such as plants is the low efficiency that has been reported so far. In maize Zhu et al. (2000 Nature Biotech. 18: 555-558) reported a conversion frequency of 1×10⁻⁴. Subsequent studies in tobacco (Kochevenko et al. 2003 Plant Phys. 132: 174-184) and rice (Okuzaki et al. 2004 Plant Cell Rep. 22: 509-512) have reported frequencies of 1×10⁻⁶ and 1×10⁻⁴ respectively. These frequencies remain too low for the practical application of TNE.

TNE using chimeric DNA:RNA oligonucleotides has been described in a variety of patent applications of Kmiec, inter alia in WO0173002, WO03/027265, WO01/87914, WO99/58702, WO97/48714, WO02/10364. In WO 01/73002 it is contemplated that the low efficiency of gene alteration obtained using unmodified DNA oligonucleotides is largely believed to be the result of degradation of the donor oligonucleotides by nucleases present in the reaction mixture or the target cell. To remedy this problem, it is proposed to incorporate modified nucleotides that render the resulting oligonucleotides resistant against nucleases. Typical examples include nucleotides with phosphorothioate linkages or 2′-O-methyl-analogs. These modifications are preferably located at the ends of the oligonucleotide, leaving a central DNA domain surrounding the mismatch nucleotide. Furthermore, the publication stipulates that specific chemical interactions are involved between the converting oligonucleotide and the proteins involved in the conversion. The effect of such chemical interactions to produce nuclease resistant termini using modifications other than phosphorothioate linkages or 2′-O-methyl analogue incorporation in the oligonucleotide is impossible to predict because the proteins involved in the alteration process and their chemical interaction with the oligonucleotide substituents are not yet known and, according to the inventors of WO0173002, cannot be predicted.

TNE using modified single stranded oligonucleotides has been described in the patent application WO 02/26967. This application demonstrates the effect of modified nucleotides, conferring improved nuclease resistance on the oligonucleotide, on the efficiency of TNE using the cell free system. The application then goes on to show that the modified nucleotides identified using the cell free system, when incorporated in a ss oligonucleotide, also enhance TNE at a mammalian chromosomal target. This confirms that information gained in the in vitro cell free assay is applicable in vivo at chromosomal loci. This application also claims that several modified nucleotides, e.g. C5-propyne pyrimidines, but not locked nucleic acids, can also be used to improve the TNE efficiency. The authors state (pg 16) that “the modifications incorporated into the oligonucleotide will not alter cellular functions that are responsible for biological activity, in this case recombination and repair activity”. In contrast, we demonstrate in this application that several modified nucleotides showing enhanced binding activity named in WO 02/26967 are biologically inactive in the TNE process and so a prediction of the usefulness of modified nucleotides in the TNE process cannot be made solely on the basis of enhanced binding affinity.

As the efficiency of the current methods of TNE is relatively low (as stated previously, between 10⁻⁶ and 10⁻⁴, despite reported high delivery rates of the oligonucleotide of 90%) there is a need in the art to come to methods for TNE that are more efficient. Accordingly, the present inventors have set out to improve on the existing TNE technology.

### DESCRIPTION OF THE INVENTION

The present inventors have now found that by incorporating a combination of modified nucleotides into the donor oligonucleotide for TNE that are capable of binding more strongly to the acceptor DNA than the corresponding unmodified nucleotides like A, C, T, or G, the rate of TNE can be increased significantly. Without being bound by theory, the present inventors believe that by the incorporation of modified nucleotides into the donor oligonucleotide, the donor oligonucleotide binds more strongly to the acceptor DNA and, hence increases the ratio of TNE.

Furthermore, the present inventors have found that the ability of a modified nucleotide to enhance TNE relies on two important properties, (1) its increased binding affinity to its target compared with normal unmodified DNA and, (2) its biological activity for the TNE process. Antisense technology also utilizes oligonucleotides to induce an effect in the cell. In this case the oligonucleotides are designed to bind to the complementary target mRNA. This DNA:RNA hybrid is recognized by RNaseH which cuts the RNA strand of the hybrid, inhibiting gene expression. The oligonucleotide characteristics, improved binding affinity and enhanced nuclease resistance, necessary to obtain an effective antisense response are also characteristics that have now been found important to enhance the frequency of TNE. Many modified nucleotides have been reported in literature that show either enhanced binding affinity and/or nuclease resistance, but the crucial step is to demonstrate that oligonucleotides containing such modified nucleotides remain biologically active for the TNE process.

The present inventors have used the cell free system to screen many modified nucleotides for their ability to enhance TNE and have found that this cannot be predicted by study of the physical properties of the modified nucleotides. In addition, the present inventors found that many modified nucleotides used to enhance the properties of antisense oligonucleotides in fact inhibit TNE in the cell free system. Thus the present inventors have identified modified nucleotides that are specific for improving the efficiency of TNE itself. Furthermore it is believed that the present oligonucleotides present advantageous stereochemical and spatial configurations.

The present inventors have found that oligonucleotides containing one or more LNAs at positions close to, but not (directly) adjacent to the mismatch, i.e. located at a distance of at least one nucleotide from the mismatch, in combination with C7-propyne modified purine and/or C5-propyne modified pyrimidines (together indicated as propynylated nucleotides) improves the efficiency of TNE to an hitherto unexpected extent, in particular improves the efficiency of in vivo TNE, i.e. not in a cell free system, but for instance in a protoplast system.

To this end, the effect of using oligonucleotides incorporating one or more LNAs and one or more propynylated nucleotides at various positions in the oligonucleotide on the frequency of TNE in the cell free system has been investigated. The TNE activity of such oligonucleotides was compared with the TNE activity of oligonucleotides made up of normal DNA or of oligonucleotides that were only modified with LNAs or propynylated nucleotides. It was found that oligonucleotides containing one or more LNAs at positions removed at least one nucleotide from the mismatch in combination with an increasing amount of propynylated nucleotides increased the TNE efficiency for both substitutions and insertions in the cell free assay to a level hitherto unobserved. In particular advantageous effects were achieved with insertions of nucleotides, i.e. inserting one or more nucleotides at a given position and in particular in in vivo systems, such as protoplast systems, the efficiency was markedly enhanced.

It was further found that this enhancement could also be improved by varying the number and positions of the LNAs observed when modified oligonucleotides were used for TNE in tomato leaf protoplasts where they gave an unprecedented increase in the oligonucleotide. Such as whereby the LNAs are positioned at least 2 nucleotides apart, preferably at least 3, more preferably at least 4.

Furthermore it was found introduction of nucleotide changes in the tomato genome. This demonstrates that the improvement observed was locus-independent, indicating that oligonucleotides of the invention with modified nucleotides at particular positions compared to the mismatch are capable of providing enhanced frequencies of TNE in a species-independent manner, such as in plant and animal cells.

The present invention is thus based on the inventive consideration that the desired targeted nucleotide exchange can be achieved by the use of partly (i.e. at most 50%, preferably at most 40%) LNA modified oligonucleotides that further contain propynylated oligonucleotides. The location, type and amount of modification of the oligonucleotide can be varied within limits as will be disclosed herein below.

The present invention thus, in one aspect provides LNA modified and propynylated oligonucleotides. The thus modified, ss-oligonucleotides can be used to introduce specific genetic changes in plant and animal or human cells. The invention is applicable in the field of biomedical research, agriculture and to construct specifically mutated plants and animals, including humans. The invention is also applicable in the field of medicine and gene therapy.

The sequence of an oligonucleotide of the invention is homologous to the target strand except for the part that contains a mismatch base that introduces the base change in the target strand. The mismatched base is introduced into the target sequence. By manipulating the modification (compared to conventional A, C, T, or G) of the nucleotides, and more in particular, by manipulating the location and amount of modification of the oligonucleotide that introduces the mismatch, the efficiency (or the degree of successful nucleotide changes at the desired position in the DNA duplex) can be improved.

Another aspect of the invention resides in a method for the targeted alteration of a parent DNA strand (first strand, second strand) by contacting the parent DNA duplex with an oligonucleotide that contains at least one mismatch nucleotide compared to the parent strand, wherein the donor oligonucleotide contains a section that is modified with LNA at particular positions to have a higher binding capacity than the parent (acceptor) strand in the presence of proteins that are capable of targeted nucleotide exchange.

Thus, the inventive gist of the invention lies in the improvement in the binding capacity of the oligonucleotide (sometimes referred to as the donor) with modified nucleotides relative to the unmodified oligonucleotide, whereby the LNA modification is located at one or more positions that are not adjacent to the mismatch and whereby further propynylated nucleotides are incorporated in the oligonucleotide, typically, at the positions that are not already LNA modified and not at the position of the mismatch.

Advantageous results have also been achieved with insertions into DNA stretches using the oligonucleotides according to the invention with combined LNA and propyne modifications. It was particularly surprising to note that other modified oligonucleotides that are known in themselves to improve binding efficiency, were not as effective in TNE as the present combination of LNA and propyne-modified oligonucleotides.

### DETAILED DESCRIPTION OF THE INVENTION

In one aspect the invention relates to an oligonucleotide for targeted alteration of a duplex DNA sequence, the duplex DNA sequence containing a first DNA sequence and a second DNA sequence which is the complement of the first DNA sequence, the oligonucleotide comprising a domain that is capable of hybridising to the first DNA sequence, which domain comprises at least one mismatch with respect to the first DNA sequence, and wherein the oligonucleotide comprises at least one section that contains at least two modified nucleotides having a higher binding affinity compared to naturally occurring A, C, T or G nucleotides, wherein
at least one modified nucleotide is a LNA that is positioned at a distance of at least one nucleotide from the at least one mismatch and wherein, optionally, the oligonucleotide contains at most about 50% LNA modified nucleotides; and
at least one modified nucleotide is a C7-propyne purine or a C5-propyne pyrimidine.

In one aspect, the invention pertains to a modified oligonucleotide for targeted alteration of a duplex DNA sequence. The duplex DNA sequence contains a first DNA sequence and a second DNA sequence. The second DNA sequence is the complement of the first DNA sequence and pairs to it to form a duplex. The oligonucleotide comprises a domain that comprises at least one mismatch with respect to the duplex DNA sequence to be altered. Preferably, the domain is the part of the oligonucleotide that is complementary to the first strand, including the at least one mismatch.

Preferably, the mismatch in the domain is with respect to the first DNA sequence. The oligonucleotide comprises a section that is modified with at least one LNA and a section that is modified with at least one propynylated nucleotide to have a higher binding affinity than the (corresponding part of the) second DNA sequence whilst retaining biological activity. Preferably the at least one modified LNA nucleotide is positioned at a distance of at least one nucleotide from the at least one mismatch, more preferably the oligonucleotide contains at most about 50% LNA modified nucleotides in addition to the at least one propynylated nucleotide.

The domain that contains the mismatch and the sections containing the modified nucleotide(s), whether LNA or propynylated, respectively, may be overlapping. Thus, in certain embodiments, the domain containing the mismatch is located at a different position on the oligonucleotide than the section of which the modification is considered. In certain embodiments, the domain incorporates one or more sections. In certain embodiments, sections can incorporate the domain. In certain embodiments, the domain and the sections may be located at the same position on the oligonucleotide and have the same length i.e. the sections coincide in length and position. In certain embodiments, there can be more than one section within a domain.

For the present invention, this means that the part of the oligonucleotide that contains the mismatch which is to alter the DNA duplex can be located at a different or shifted position from the part of the oligonucleotide that is modified. In particular, in certain embodiments wherein the cell's repair system (or at least the proteins involved with this system, or at least proteins that are involved in TNE) determines which of the strands contain the mismatch and which strand is to be used as the template for the correction of the mismatch.

LNA Variations

A Locked Nucleic Acid (LNA) is a DNA analogue with very interesting properties for use in antisense gene therapy. LNAs are bicyclic and tricyclic nucleoside and nucleotide analogues and the oligonucleotides that contain such analogues. The basic structural and functional characteristics of LNAs and related analogues are disclosed in various publications and patents, including WO 99/14226, WO 00/56748, WO00/66604, WO 98/39352, U.S. Pat. No. 6,043,060, and U.S. Pat. No. 6,268,490, all of which are incorporated herein by reference in their entireties.

Specifically, it combines the ability to discriminate between correct and incorrect targets (high specificity) with very high bio-stability (low turnover) and unprecedented affinity (very high binding strength to target). In fact, the affinity increase recorded with LNA leaves the affinities of all previously reported analogues in the low-to-modest range.

LNA is an RNA analogue, in which the ribose is structurally constrained by a methylene bridge between the 2′-oxygen and the 4′-carbon atoms. This bridge restricts the flexibility of the ribofuranose ring and locks the structure into a rigid bicyclic formation. This so-called N-type (or 3′-endo) conformation results in an increase in the Tₘ of LNA containing duplexes, and consequently higher binding affinities and higher specificities. NMR spectral studies have actually demonstrated the locked N-type conformation of the LNA sugar, but also revealed that LNA monomers are able to twist their unmodified neighbour nucleotides towards an N-type conformation. Importantly, the favourable characteristics of LNA do not come at the expense of other important properties as is often observed with nucleic acid analogues.

LNA can be mixed freely with all other chemistries that make up the DNA analogue universe. LNA bases can be incorporated into oligonucleotides as short all-LNA sequences or as longer LNA/DNA chimeras. LNAs can be placed in internal, 3′ or 5″-positions. However, due to their rigid bicyclic conformations, LNA residues sometimes disturb the helical twist of nucleic acid strands. It is hence generally less preferred to design an oligonucleotide with two or more adjacent LNA residues. Preferably, the LNA residues are separated by at least one (modified) nucleotide that does not disturb the helical twist, such as a conventional nucleotide (A, C, T, or G).

The originally developed and preferred LNA monomer (the β-D-oxy-LNA monomer) has been modified into new LNA monomers. The novel α-L-oxy-LNA shows superior stability against 3′ exonuclease activity, and is also more powerful and more versatile than β-D-oxy-LNA in designing potent antisense oligonucleotides. Also xylo-LNAs and L-ribo LNAs can be used, as disclosed in WO9914226, WO00/56748, WO00/66604. In the present invention, any LNA of the above types may be effective in achieving the goals of the invention, i.e. improved efficiency of TNE, with a preference for β-D-LNA analogues.

In the art on TNE, LNA modification has been listed amongst a list of possible oligonucleotide modifications as alternatives for the chimeric molecules used in TNE. However, there is no indication in the art thus far that suggests that LNA modified single-stranded DNA oligonucleotides enhances TNE efficiency significantly to the extent that has presently been found when the LNA is positioned at least one nucleotide away from the mismatch and/or the oligonucleotide does not contain more than about 50% (rounded to the nearest whole number of nucleotides) LNAs.

In certain embodiments, the oligonucleotide comprises a section that contains at least one, preferably at least 2, more preferably 2 LNA modified nucleotide(s). In certain embodiments, the section on the oligonucleotide can contain more than 2, 3, 4, 5, 6, 7, 8, 9, or 10 LNA modified nucleotides.

In certain embodiments, the at least one LNA is positioned at a distance of at most 10 nucleotides, preferably at most 8 nucleotides, more preferably at most 6 nucleotides, even more preferably at most 4, 3, or 2 nucleotides from the mismatch. In a more preferred embodiment the at least one LNA is positioned at a distance of 1 nucleotide from the mismatch, i.e. one nucleotide is positioned between the mismatch and the LNA. In certain embodiments relating to oligonucleotides containing more than one LNA, at least two of the LNAs are located at a distance of at least one nucleotide from the mismatch. In a preferred embodiment, LNAs are not located adjacent to each other but are spaced apart by at least one nucleotide, preferably two or three nucleotides. In certain embodiments, in the case of two or more (even numbers of) LNA modifications of the oligonucleotide, the modifications are spaced at (about) an equal distance from the mismatch. In other words, preferably the LNA modifications are positioned symmetrically around the mismatch. For example, in a preferred embodiment, two LNAs are positioned symmetrically around the mismatch at a distance of 1 nucleotide from the mismatch (and 3 nucleotides from each other), i.e . . . (LNA)-N-(Mismatch)-N-(LNA) . . . .

In certain embodiments, at most 40% of the modified nucleotides of the oligonucleotide are LNA derivatives, i.e. the conventional A, C, T or G is replaced by its LNA counterpart, preferably at most 30%, more preferably at most 25%, even more preferably at most 20%, and most preferably at most 10%.

In certain embodiments, more than one mismatch can be introduced, either simultaneously or successively. The oligonucleotide can accommodate more than one mismatch on either adjacent or removed locations on the oligonucleotide. To this end, the oligonucleotide can be adapted to accommodate a second set of LNAs that follow the principles outlined herein, provided they do not interfere with each other's improved binding capacity or retained biological activity due to the particular conformation of the LNAs in the oligonucleotide, i.e. preferably spaced around the mismatch at a distance of 1 nucleotide from the mismatch. In certain embodiments the oligonucleotide can comprise two, three, four or more mismatch nucleotides which may be adjacent or remote (i.e. non-adjacent). The oligonucleotide can comprise further domains and sections to accommodate this, and in particular can comprise several sections. In certain embodiments, the oligonucleotide may incorporate a potential insert that is to be inserted in the acceptor strand. Such an insert may vary in length from more than five up to 100 nucleotides. In a similar way in certain embodiments, deletions can be introduced of similar length variations (from 1 to 100 nucleotides).

The delivery of the oligonucleotide can be achieved via electroporation or other conventional techniques that are capable of delivering either to the nucleus or the cytoplasm. In vitro testing of the method of the present invention can be achieved using the Cell Free system as is described i.a. in WO01/87914, WO03/027265, WO99/58702, WO01/92512.

Propyne Modifications

In certain embodiments, the oligonucleotide comprises a section that contains at least one, preferably at least 2, more preferably at least 3 propyne modified nucleotide(s), independently selected from amongst C7 purine and/or C5 pyrimidine. In certain embodiments, the section on the oligonucleotide can contain more than 4, 5, 6, 7, 8, 9, or 10 propyne modified nucleotides. In certain embodiments the section is fully modified, i.e. all pyrimidines in the oligonucleotide carry a C5-propyne substitution and/or all purines carry a C7-propyne substitution, the LNA modification can then be located at the sides of the section. In certain embodiments there are sections that are LNA modified and sections that are propynyl modified. In certain embodiments the section contains both LNA and propyne modified nucleotides. In certain embodiments, the oligonucleotide comprises a section that contains at least one, preferably at least 2, more preferably at least 3 propyne modified nucleotide(s). In certain embodiments, the section on the oligonucleotide can contain more than 4, 5, 6, 7, 8, 9, or 10 propyne modified nucleotides. In certain embodiments the section is fully modified, i.e. all pyrimidines in the oligonucleotide carry a C5-propyne substitution and/or all purines carry a C7-propyne substitution. In certain embodiments all purines may be propynylated and the pyrimidines may be substituted by LNAs or vice versa. In certain embodiments, at least 10% of the nucleotides in the oligonucleotide is replaced by its propynylated counterpart. In certain embodiments at least 25, more preferably at least 50%, even more preferably at least 75% and in some cases it is preferred that at least 90% of the nucleotides are replaced by their propynylated counterparts.

Oligonucleotides containing pyrimidine nucleotides with a propynyl group at the C5 position form more stable duplexes and triplexes than their corresponding pyrimidine derivatives. Purine with the same propyne substituent at the 7-position form even more stable duplexes and are hence preferred. Thus, in certain preferred embodiments, efficiency was further increased through the use of 7-propynyl purine nucleotides (7-propynyl derivatives of 8-aza-7-deaza-2′-deoxyguanosine and 8-aza-7-deaza-2′-deoxyadenine) which enhance binding affinity to an even greater degree than C5-propyne pyrimidine nucleotides. Such nucleotides are disclosed inter alia in He & Seela, 2002 Nucleic Acids Res. 30: 5485-5496.

In certain embodiments, more than one mismatch can be introduced, either simultaneously or successively. The oligonucleotide can accommodate more than one mismatch on either adjacent or removed locations on the oligonucleotide. In certain embodiments the oligonucleotide can comprise two, three, four or more mismatch nucleotides which may be adjacent or remote (i.e. non-adjacent). The oligonucleotide can comprise further domains and sections to accommodate this, and in particular can comprise several sections. In certain embodiments, the oligonucleotide may incorporate a potential insert that is to be inserted in the acceptor strand. Such an insert may vary in length from more than five up to 100 nucleotides. In a similar way in certain embodiments, deletions can be introduced of similar length variations (from 1 to 100 nucleotides).

In certain advantageous embodiments of the invention, nucleotide insertions have been achieved using the oligonucleotides of the invention. In certain embodiments, the oligonucleotide may incorporate a potential insert that is to be inserted in the acceptor strand. Such an insert may vary in length from 1, 2, 3, 4, 5 up to 100 nucleotides. In a similar way in certain embodiments, deletions can be introduced of similar length variations (from 1 to 100 nucleotides).

In certain embodiments, at least 10% of the nucleotides in the oligonucleotide is replaced by its propynylated counterpart. In certain embodiments at least 25, more preferably at least 50%, even more preferably at least 75% and in some cases it is preferred that at least 90% of the nucleotides are replaced by their propynylated counterparts.

A propynyl group is a three carbon chain with a triple bond. The triple bond is covalently bound to the nucleotide basic structure which is located at the C⁵ position of the pyrimidine and at the 7-position of the purine nucleotide (FIG. 2). Both cytosine and uracil (replacing thymidine on the oligonucleotide) can be equipped with C5-propynyl group, resulting in C⁵-propynyl-cytosine and C⁵-propynyl-uracil, respectively. A single C⁵-propynyl-cytosine residue increases the Tₘ by 2.8° C., a single C⁵-propynyl-uracil by 1.7° C. (Froehler et al. 1993 Tetrahedron Letters 34: 1003-6; Lacroix et al. 1999 Biochemistry 38: 1893-1901; Ahmadian et al. 1998 Nucleic Acids Res. 26: 3127-3135; Colocci et al. 1994 J. Am. Chem. Soc 116: 785-786). This is attributed to the hydrophobic nature of 1-propyne groups at the C5 position and it also allows better stacking of the bases since the propyne group is planar with respect to the heterocyclic base.

The improved binding properties of oligonucleotides containing C5-propyne substituted pyrimidine groups has been exploited to alter a cellular process. An antisense oligonucleotide containing C5-propyne groups forms a more stable duplex with its target mRNA, leading to an increase in the inhibition of gene expression (Wagner et al. 1993 Science 260: 1510-1513; Flanagan et al. 1996 Nature Biotech. 14: 1139-1145; Meunier et al. 2001 Antisense & Nucleic Acid Drug Dev. 11: 117-123). Furthermore, these experiments demonstrate that such oligonucleotides are biologically active and that they can be tolerated by the cell. In the art on TNE, C5-propyne pyrimidine modification has been listed amongst a list of possible oligonucleotide modifications as alternatives for the chimeric molecules used in TNE. However, there is no indication in the art thus far that suggests that C5 and or C7 propyne modified single-stranded DNA oligonucleotides enhances TNE efficiency significantly to the extent that has presently been found in combination with LNA modification.

Oligo Design

In a further aspect of the invention, the design of the oligonucleotide can be achieved by:
determining the sequence of the acceptor strand, or at least of a section of the sequence around the nucleotide to be exchanged. This can typically be in the order of at least 10, preferably 15, 20, 25 or 30 nucleotides adjacent to the mismatch or desired position of the insert, preferably on each side of the mismatch, (for example GGGGGGXGGGGGG, wherein X is the mismatch or insert position);
designing a donor oligonucleotide that is complementary to one or both the sections adjacent to the mismatch and contains the desired nucleotide to be exchanged (for example CCCCCCYCCCCCC);
providing (e.g. by synthesis) the donor oligonucleotide with LNA and propyne modifications at desired positions. Modifications may vary widely, depending on the circumstances. Examples are CCC^{m}CC^{m}CYCC^{m}CCC^{m}C, CCC^{m}CCCYCCC^{m}CCC, CCCCCCYCCC^{m}C^{m}C^{m}C^{m}, C^{m}C^{m}C^{m}C^{m}C^{m}CYCCCCCC, CCCCC^{m}CYCCC^{m}CCCCC, and so on, wherein C^{m} stands for a LNA or propyne modified nucleotide residue. For a different acceptor sequence, e.g. ATGCGTACXGTCCATGAT, corresponding donor oligonucleotides can be designed, e.g. TACGCALGYCLGGTACTA (L=LNA or propyne) with modification as variable as outlined hereinbefore.
subjecting the DNA to be modified with the donor oligonucleotide in the presence of proteins that are capable of targeted nucleotide exchange, for instance, and in particular, proteins that are functional in the mismatch repair mechanism of the cell.

Without being bound by theory, improved binding affinity is thought to increase the likelihood that an oligonucleotide finds and remains bound to its target, thus improving the TNE efficiency. Many different chemical modifications of the sugar backbone or the base confer improved binding affinity. The present inventors however, chose to focus on LNA modified oligonucleotides and found that their activity in TNE was dependent on the position in the oligonucleotide.

As used herein, the capability of the donor oligonucleotide to influence the TNE depends on the type, location and number or relative amount of modified nucleotides that are incorporated in the donor oligonucleotide. This capability can be quantified for instance by normalising the binding affinity (or the binding energy (Gibbs Free Energy)) between conventional nucleotides at 1, i.e. for both. AT and GC bindings, the binding affinity is normalised at 1. For the oligonucleotides of the present invention the Relative Binding Affinity (RBA) of each modified nucleotide is >1. This is exemplified in a formula below:

$$RBA = {\sum }_{n}^{1} RBA ( modified ) - {\sum }_{m}^{1} RBA ( unmodified ) > 0$$

Wherein RBA is the total relative binding affinity, RBA (modified) is the sum of the relative binding affinity of the modified oligonucleotide with a length of n nucleotides and RBA (unmodified) is the sum of the relative binding affinity of the unmodified oligonucleotide with a length of m nucleotides. For example, an 100 bp oligonucleotide contains 10 modifications, each with a relative binding affinity of 1.1. The total RBA then equals: RBA=[(10*1.1)+(90*1.0)]−(100*1.0)=1.

Note that the definition of RBA is in principle independent of the length of the nucleotide strand that is compared. However, when RBAs of different strands are compared it is preferred that the strands have about the same length or that sections of comparable length are taken. Note that RBA does not take into account that modifications can be grouped together on a strand. A higher degree of modification of a certain strand A compared to a strand B thus means that RBA(A)>RBA(B). For upstream and downstream sections, corresponding (local) RBA values may be defined and used. To accommodate the effect of the position of the modified nucleotide a weighing factor can be introduced into the RBA value. For instance, the effect of a modified nucleotide on the donor oligonucleotide adjacent to the mismatch can be larger than that of a modified nucleotide that is located at a distance five nucleotides removed from the mismatch. In the context of the present invention, RBA (Donor)>RBA (Acceptor).

In certain embodiments, the RBA value of the Donor may be at least 0.1 larger than the RBA of the Acceptor. In certain embodiments, the RBA value of the Donor may be at least 0.2, 0.3, 0.4, 0.5, 0.6, 0.7, 0.8, 0.9, 1.0, 1.5, 2.0, 2.5 larger than the RBA of the Acceptor. RBA values can be derived from conventional analysis of the modified binding affinity of the nucleotide, such as by molecular modelling, thermodynamic measurements etc. Alternatively they can be determined by measurement of Tm differences between modified and unmodified strands. Alternatively, the RBA can be expressed as the difference in Tm between the unmodified and the modified strand, either by measurement or by calculation using conventional formulates for calculating the Tm of a set of nucleotides, or by a combination of calculation and measurements.

The donor oligonucleotides according to the invention may contain further modifications to improve the hybridisation characteristics such that the donor exhibits increased affinity for the target DNA strand so that intercalation of the donor is promoted. The donor oligonucleotide can also be further modified to become more resistant against nucleases, to stabilise the triplex or quadruplex structure. Modification of the LNA modified donor oligonucleotides of the invention can comprise phosphorothioate modification, 2-OMe substitutions, the use of further types of LNAs at the 3 and/or 5′ termini of the oligonucleotide, PNAs (Peptide nucleic acids), ribonucleotide and other bases that modifies, preferably enhances, the stability of the hybrid between the oligonucleotide and the acceptor strand.

Particularly useful among such modifications are PNAs, which are oligonucleotide analogues where the deoxyribose backbone of the oligonucleotide is replaced by a peptide backbone. One such peptide backbone is constructed of repeating units of N-(2-aminoethyl) glycine linked through amide bonds. Each subunit of the peptide backbone is attached to a nucleobase (also designated “base”), which may be a naturally occurring, non-naturally occurring or modified base. PNA oligomers bind sequence specifically to complementary DNA or RNA with higher affinity than either DNA or RNA. Accordingly, the resulting PNA/DNA or PNA/RNA duplexes have higher melting temperatures (Tm). In addition, the Tm of the PNA/DNA or PNA/RNA duplexes is much less sensitive to salt concentration than DNA/DNA or DNA/RNA duplexes. The polyamide backbone of PNAs is also more resistant to enzymatic degradation. The synthesis of PNAs is described, for example, in WO 92/20702 and WO 92/20703, the contents of which are incorporated herein by reference in their entireties. Other PNAs are illustrated, for example, in WO93/12129 and U.S. Pat. No. 5,539,082, issued Jul. 23, 1996, the contents of which are incorporated herein by reference in their entireties. In addition, many scientific publications describe the synthesis of PNAs as well as their properties and uses. See, for example, Patel, Nature, 1993, 365, 490; Nielsen et al., Science, 1991, 254, 1497; Egholm, J. Am. Chem. Soc., 1992, 114, 1895; Knudson et al., Nucleic Acids Research, 1996, 24, 494; Nielsen et al., J. Am. Chem. Soc., 1996, 118, 2287; Egholm et al., Science, 1991, 254, 1497; Egholm et al., J. Am. Chem. Soc., 1992, 114, 1895; and Egholm et al., J. Am. Chem. Soc., 1992, 114, 9677.

Useful further modifications of the oligonucleotides of the present invention are also known as Super A and Super T, obtainable from Epoch Biosciences Germany. These modified nucleotides contain an additional substituent that sticks into the major groove of the DNA where it is believed to improve base stacking in the DNA duplex.

In further embodiments, advantageous results can be achieved when, in addition to the modified oligonucleotides according to the invention, further modifications are introduced into oligonucleotide that enhance affinity of the oligonucleotide for the acceptor strand even more. Thus it has been found that LNA modified oligonucleotide according to the invention which further comprise C5-propyne modified pyrimidine and/or C7 propynyl modified purines improves the efficiency of TNE significantly.

The donor oligonucleotides of the invention can also be made chimeric, i.e. contain sections of DNA, RNA, LNA, PNA or combinations thereof.

Thus, in certain embodiments, the oligonucleotide of the invention further contains other, optionally non-methylated, modified nucleotides.

In certain embodiments, the oligonucleotide is resistant against nucleases. This may be advantageous to prevent the oligonucleotide from being degraded by nucleases and enlarges the chance that the donor oligonucleotide can find its target (acceptor molecule).

In certain embodiments of the invention, the nucleotide in the oligonucleotide at the position of the mismatch can be modified. Whether or not the mismatch can be modified will depend to a large extent on the exact mechanism of the targeted nucleotide exchange or of the cell's DNA repair mechanism using the difference in affinity between the donor and acceptor strands. The same holds for the exact location of the other modified positions in the neighbourhood or vicinity of the mismatch. However, based on the disclosure presented herein, such an oligonucleotide can be readily designed and tested, taking into account the test procedures for suitable oligonucleotides as described herein elsewhere. In certain embodiments, the nucleotide at the position of the mismatch is not modified. In certain embodiments, modification is at a position one nucleotide away from to the mismatch, preferably 2, 3, 4, 5, 6 or 7 nucleotides away from the mismatch. In certain embodiments, modification is located at a position downstream from the mismatch. In certain embodiments, modification is located at a position upstream from the mismatch. In certain embodiments, the modification is located from 10 bp to 10 kB from the mismatch, preferably from 50 to 5000 bp, more preferably from 100 to 500 from the mismatch.

The oligonucleotides that are used as donors can vary in length but generally vary in length between 10 and 500 nucleotides, with a preference for 11 to 100 nucleotides, preferably from 15 to 90, more preferably from 20 to 70 most preferably from 30 to 60 nucleotides.

In one aspect, the invention pertains to a method for the targeted alteration of a duplex acceptor DNA sequence, comprising combining the duplex acceptor DNA sequence with a donor oligonucleotide, wherein the duplex acceptor DNA sequence contains a first DNA sequence and a second DNA sequence which is the complement of the first DNA sequence and wherein the donor oligonucleotide comprises a domain that comprises at least one mismatch with respect to the duplex acceptor DNA sequence to be altered, preferably with respect to the first DNA sequence, and wherein a section of the donor oligonucleotide is modified with at least one LNA and at least one propynylated nucleotide as to express a higher degree of affinity to the first DNA sequence compared to an unmodified nucleotide at that position in the oligonucleotide, in the presence of proteins that are capable of targeted nucleotide exchange, wherein the LNA is positioned at a distance of at least one nucleotide vis-à-vis the mismatch.

The invention is, in its broadest form, generically applicable to all sorts of organisms such as humans, animals, plants, fish, reptiles, insects, fungi, bacteria and so on. The invention is applicable for the modification of any type of DNA, such as DNA derived from genomic DNA, linear DNA, artificial chromosomes, nuclear chromosomal DNA, organelle chromosomal DNA, BACs, YACs. The invention can be performed in vivo as well as ex vivo.

The invention is, in its broadest form, applicable for many purposes for altering a cell, correcting a mutation by restoration to wild type, inducing a mutation, inactivating an enzyme by disruption of coding region, modifying bioactivity of an enzyme by altering coding region, modifying a protein by disrupting the coding region.

The invention also relates to the use of oligonucleotides essentially as described hereinbefore, for altering a cell, correcting a mutation by restoration to wild type, inducing a mutation, inactivating an enzyme by disruption of coding region, modifying bioactivity of an enzyme by altering coding region, modifying a protein by disrupting the coding region, mismatch repair, targeted alteration of (plant) genetic material, including gene mutation, targeted gene repair and gene knockout

The invention further relates to kits, comprising one or more oligonucleotides as defined herein elsewhere, optionally in combination with proteins that are capable of inducing targeted mutagenesis and in particular that are capable of TNE.

The invention further relates to modified genetic material obtained by the method of the present invention, to cells and organisms that comprise the modified genetic material, to plants or plant parts that are so obtained.

The delivery of the oligonucleotide can be achieved via electroporation or other conventional techniques that are capable of delivering either to the nucleus or the cytoplasm. In vitro testing of the method of the present invention can be achieved using the Cell Free system as is described i.a. in WO01/87914, WO03/027265, WO99/58702, WO01/92512.

The invention is, in its broadest form, applicable for many purposes for altering a cell, correcting a mutation by restoration to wild type, inducing a mutation, inactivating an enzyme by disruption of coding region, modifying bioactivity of an enzyme by altering coding region, modifying a protein by disrupting the coding region.

The invention also relates to the use of oligonucleotides essentially as described hereinbefore, for altering a cell, correcting a mutation by restoration to wild type, inducing a mutation, inactivating an enzyme by disruption of coding region, modifying bioactivity of an enzyme by altering coding region, modifying a protein by disrupting the coding region, mismatch repair, targeted alteration of (plant) genetic material, including gene mutation, targeted gene repair and gene knockout

The invention further relates to kits, comprising one or more oligonucleotides as defined herein elsewhere, optionally in combination with proteins that are capable of inducing targeted mutagenesis and in particular that are capable of TNE.

The invention further relates to modified genetic material obtained by the method of the present invention, to cells and organisms that comprise the modified genetic material, to plants or plant parts that are so obtained.

The invention relates in particular to the use of the TNE method using the LNA and propyne-modified oligonucleotides of the invention to provide for herbicide resistance in plants. In particular the invention relates to plants that have been provided with resistance against herbicides, in particular sulfonylurea herbicides (e.g. chlorsulfuron) and glyphosate.

The invention further relates to a method for increasing targeted nucleotide exchange efficiency of a duplex DNA, the method comprising the steps of (a) obtaining an oligonucleotide comprising a domain that is capable of hybridizing to a first DNA sequence of said duplex, wherein said domain comprises: (i) at least one mismatch with respect to the first DNA sequence; and (ii) at least one modified nucleotide having an increased binding affinity, and (b) decreasing the distance between said modified nucleotide and said mismatch to about 8 or fewer nucleotides; and (c) recovering an oligonucleotide for use in targeted nucleotide exchange. This method is based on the observations that the limitations given in the prior art for the oligonucleotide, i.e. the requirement that the distance between the mismatch and the oligonucleotide must be at least 8 nucleotides is not a requirement of the oligonucleotides of the present invention. As can be seen from the appended examples, oligonucleotides that contain one or more modified nucleotides are more efficient in TNE than the unmodified oligonucleotide.

The invention further relates to an oligonucleotide for targeted alteration of a duplex DNA, wherein said oligonucleotide comprises a domain that is capable of hybridizing to a first DNA sequence of said duplex and said domain comprises: (a) at least one mismatch with respect to the first DNA sequence; (b) at least one section comprising at least one modified nucleotide having an increased binding affinity, wherein said modified nucleotide is a LNA or a propyne modified nucleotide (as described herein elsewhere, wherein said modified nucleotide is positioned at most 8 nucleotides from said mismatch. In certain embodiments the sections comprises two or more modified nucleotides selected independently from amongst a LNA and a propyne modified nucleotide as described herein elsewhere. In certain embodiments, the LNA is positioned at least one but not more than 8 nucleotides from the mismatch. In certain embodiments, the modified nucleotide is positioned at most 8 nucleotides from said mismatch. In certain embodiments, the modified nucleotide is positioned at most 6 nucleotides from said mismatch. In certain embodiments, the modified nucleotide is positioned at most 4 nucleotides from said mismatch. In certain embodiments, the modified nucleotide is positioned at most 2 nucleotides from said mismatch. In certain embodiments, the oligonucleotide comprises domain that is capable of hybridizing to the first DNA sequence of the duplex, wherein said domain comprises 2 LNAs. Furthermore, An oligonucleotide for targeted nucleotide exchange of a duplex DNA, wherein said oligonucleotide comprises (a) a modified nucleotide; and (b) a mismatch with respect to a strand of said duplex DNA, wherein said modified nucleotide is positioned about 1 nucleotide away from said mismatch.

### DESCRIPTION OF THE FIGURES

FIG. 1: Schematic representation of targeted nucleotide exchange. An acceptor duplex DNA strand containing a nucleotide that is to be exchanged (X) is brought into contact with a LNA and C5-propyne pyrimidine modified donor oligonucleotide (schematically given as NNN^{m}NNN^{m}YNN^{m}NN^{m}) containing the nucleotide to be inserted (Y). The acceptor/donor structure is subjected to or brought into contact with an environment that is capable of TNE or at least with proteins that are capable of performing TNE, such as are known as the cell-free enzyme mixture or a cell-free extract (see i.a. WO99/58702, WO01/73002).

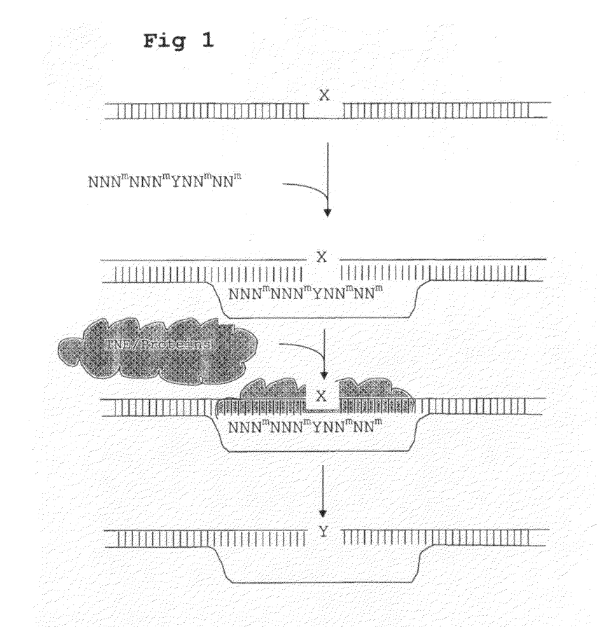

FIG. 2: Chemical structures of 5-propynyl-deoxyuracil, 5-propynyl-deoxycytosine, 2′-Deoxy-7-propynyl-7-deaza-adenosine and the 2′deoxy-7-propynyl-deaza-guanosine and locked nucleic acids.

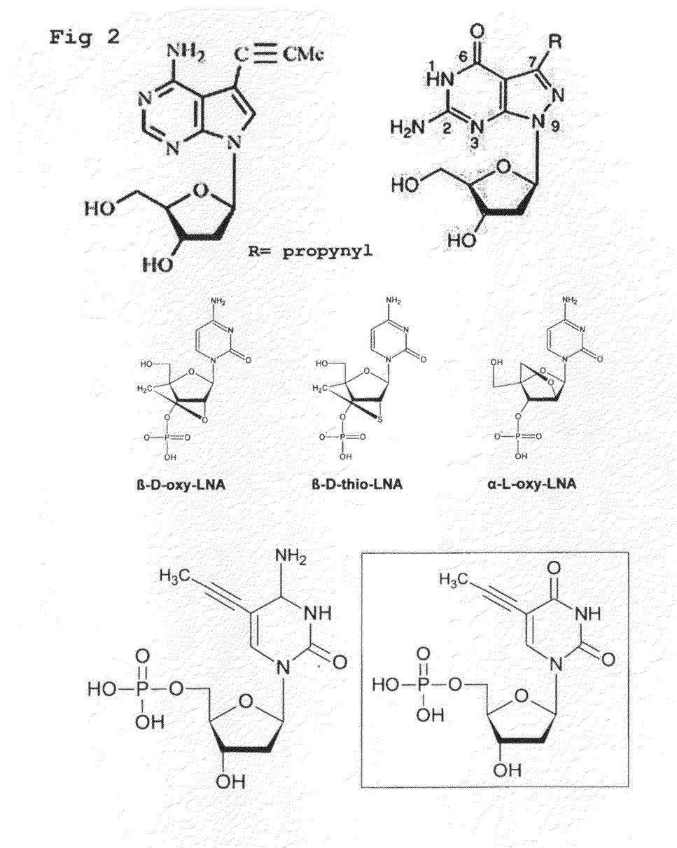

FIG. 3: Sequence analysis of the ALS P186/184 codon amplified from herbicide resistant tomato calli. Individual PCR products were cloned and sequenced in this instance.

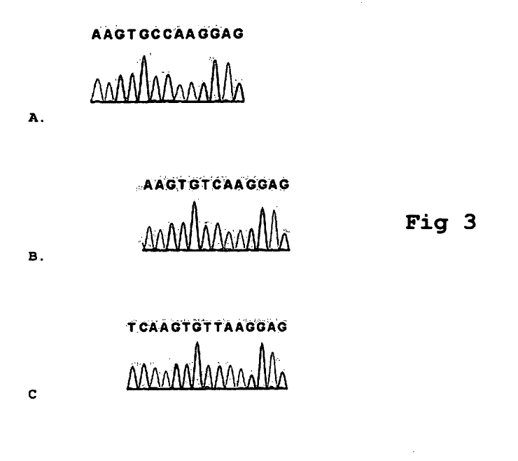

### EXAMPLES

### Example 1

### TNE in the Cell Free System

Oligonucleotides containing C5-propyne pyrimidines, LNA nucleotide or combinations thereof were purchased from Trilink Biotech, GeneLink or Ribotask. Oligonucleotides containing other modified nucleotides were purchased from Eurogentec.

The sequences of the oligonucleotides used are shown in Table 1. The plasmid used in the experiments was a derivative of pCR2.1 (Invitrogen) that contains genes conferring both kanamycin and carbenicillin resistance. Plasmid KmY22stop has a TAT to TAG mutation at codon Y22 in the kanamycin ORF. In plasmid KmY22Δ the third nucleotide of the Y22 codon (TAT) was deleted to produce a frame shift. In both plasmids, the kanamycin ORF has been mutated at the same position. Thus, a single oligonucleotide can be tested for its efficiency to produce nucleotide substitutions or insertions by incubation with either KmY22stop or KmY22Δ respectively.

| | |
|---|---|
| Km WT | GAG AGG CTA TTC GGC TAT GAC TGG GCA |
| | CAA CAG |
| | E R L F G Y D W A |
| | Q Q |
| KmY22stop | GAG AGG CTA TTC GGC TAG GAC TGG GCA CAA |
| | CAG |
| | E R L F G * |
| KmY22Δ | GAG AGG CTA TTC GGC TA_ GAC TGG GCA CAA |
| | CAG |
| | E R L F G * |

The relevant sequence of the kanamycin ORF and the amino acids encoded are shown. The single nucleotide substitution and deletion producing a stop codon (TAG, *) were introduced as previously described (Sawano et al. 2000 Nucleic Acids Res. 28: e78). The sequences of the oligonucleotide used in the experiments are shown. The oligonucleotide binding region is underlined on the kanamycin ORF.

Cell Free Assays

Cell free assays were performed as follows. Flower buds from Arabidopsis thaliana (ecotype Col-0) were collected and ground under nitrogen. 200 μl protein isolation buffer (20 mM HEPES pH7.5, 5 mM KCl, 1.5 mM MgCl₂, 10 mM DTT, 10% (v/v) glycerol, 1% (w/v) PVP) was added. The plant debris was pelleted by centrifugation at 14 k RPM for 30 mins and the supernatant was stored at −80° C. The protein concentration was measured using the NanoOrange Kit (Molecular Probes, Inc). A typical isolation resulted in a protein concentration of approximately 3-4 μg/μl. The cell free reactions contained the following components. 1 μg plasmid DNA (KmY22stop or KmY22Δ), 100 ng of oligonucleotide, 30 μg total plant protein, 4 μl sheared salmon sperm DNA (3 μg/μl), 2 μl protease inhibitor mix (50× conc: Complete EDTA-free protease inhibitor cocktail tablets, Roche Diagnostics), 50 μl 2× cell free reaction buffer (400 mM Tris pH7.5, 200 mM MgCl₂, 2 mM DTT, 0.4 mM spermidine, 50 mM ATP, 2 mM each CTP, GTP, UTP, 0.1 mM each dNTPs and 10 mM NAD) made up to a total volume of 100 μl with water. The mixture was incubated at 37° C. for 1 hr. The plasmid DNA was then isolated as follows. 100 μl H₂O was added to each reaction to increase the volume followed by 200 μl alkaline buffered phenol (pH8-10). This was vortexed briefly and then centrifuged and 13 k rpm for 3 mins. The upper aqueous phase was transferred to a new tube and 200 μl chloroform was then added. This was vortexed briefly, spun at 13 k rpm for 3 mins and the aqueous phase transferred to a new tube. The DNA was precipitated by addition of 0.7 volume 2-propanol and the pellet resuspended in TE. To eliminate any co-purified oligonucleotide the DNA was passed over a Qiagen PCR purification column and the plasmid DNA eluted in a final volume of 30 μl. 2 μl of plasmid DNA was electroporated to 18 μl of DH10B (Invitrogen) electrocompetent cells. After electroporation the cells were allowed to recover in SOC medium for 1 hr at 37° C. After this period kanamycin was added to a concentration of 100 μg/ml and the cells were incubated for a further 3 hours. The electroporation efficiency was calculated by counting the number of colonies obtained from a 10⁻⁴ and 10⁻⁵ dilution of the electroporation plated out on carbenicillin containing medium. The TNE efficiency was calculated by dividing the number of kanamycin resistant colonies by the total number of transformed cells calculated from the number of carbenicillin resistant colonies.

### Results

Cell Free Experiments Using KmY22stop

The modified nucleotides incorporated in the oligonucleotides shown in table 1 are as follows.

Methylphosphonates (MP) are non-ionic nucleic acid analogs which contain nuclease resistant methylphosphonate linkages instead of the naturally occurring negatively charged phosphodiester bonds. They have been extensively used in antisense approaches in mammalian cells.

C-5 methylated pyrimidine deoxynucleotides (5Me-dC) are known to form more stable duplexes than their corresponding pyrimidine derivatives. For example, substitution of 5-methyl-2′-deoxycytidine (5-Me-dC) has been shown to increase the Tm by 1.3° C. per substitution. Synthesis of 5-(1-propynyl)-2′-deoxyuridine (pdU) and 5-(1-propynyl)-2′-deoxycytidine (pdC) has demonstrated that both substitutions enhance duplex stability. Substitution of methyl with 1-propyne at the C5 position of pyrimidines allows better stacking of the bases since the propyne group is planar with respect to the heterocyclic base. At the same time, propyne is more hydrophobic than methyl and this property contributes further to an increase in binding. Duplex binding is enhanced by 1.7° C. per pdU and 1.5° C. per pdC residue.

Morpholino oligonucleotides are DNA analogs in which the ribose is replaced by a morpholino moiety and phosphoroamidate intersubunit linkages are used instead of phosphodiester bonds. They are used extensively for gene knockout studies in zebrafish (Genesis, Vol 30, 2001). They have also been used to correct aberrant splicing of a mutant β-globin precursor mRNA (Lacerra et al. 2000, Proc. Natl. Acad. Sci. USA 97, 9591-9596).

Locked nucleic acid (β-D-LNA), first described by Wengel and co-workers (Koshkin et al. 1998, Tetrahedron 54, 3607-3630; Singh et al, 1998, Chem. Commun. 455-456) and by Imanishi and co-workers (Okiba et al. 1998, Tetrahedron Lett. 39, 5401-5404) are conformationally restricted nucleotide derivatives. They contain a methylene 2′-O, 4′-C linkage that reduces the conformational flexibility and confers a RNA-like C3′-endo conformation to the sugar moiety of the nucleotide (Petersen et al. 2002, J. Am. Chem. Soc. 124, 5974-5982). This greatly enhances the affinity towards DNA targets improving Tm of an oligonucleotide by 4-9° C. per introduced LNA monomer compared to unmodified duplexes (Wengel, J. 2001, In Crooke, S. T. (ed), Antisense Drug Technology: Principles, Strategies and Applications. Marcel Dekker, Inc., New York, Basle, pp. 339-357). The properties of a stereoisomer of β-D-LNA, the α-L-LNA, has also been studied. It is well known that β-D-LNA is locked in a A-form with compelentary DNA, however duplexes between α-L-LNA and DNA adopt a B form (Nielsen et al. 2002, Chem. Eur. J. 8, 3001-3009) which is the natural form of double stranded DNA. Due to their increased binding affinity, both these LNA forms have been shown to improve the antisense properties of oligonucleotides (Kurreck et al. 2002, Nuc. Acids. Res. 30, 1911-1918; Frieden et al. 2003, Nuc. Acids Res. 31, 6365-6372).

Oligonucleotides bearing the 6-chloro-2-methoxyacridine molecule at either end or within the sequence have the ability to intercalate efficiently into a double helix. This intercalator thus increases hybrid stability by providing additional binding energy. Acridine-labeled oligonucleotides have been used in applications where increased stability of oligonucleotide hybrids is crucial. Addition of the dye to an oligonucleotide 3′ terminus also protects the oligonucleotide from exonuclease degradation.

The 2-amino adenine binding to thymine is intermediate in stability between A:T and G:C base pair stability due to the formation of an additional hydrogen bond.

The 2′-O-methyl nucleotides, such as 2′-O-Me inosine, are resistant to a variety of ribo- and deoxyribonucleases and also form more stable hybrids with complementary sequences.

TABLE 1

TNE efficiency of oligonucleotides containing modified oligonucleotides in a cell
free assay.

| | | | | |
|---|---|---|---|---|
| SEQ ID # | Oligonucleotide | Sequence | Modification, position | TNE eff. |

| | | | | |
|---|---|---|---|---|
| 1 | 1 | tgtgcccagtcgtagccgaatagc | None | 1 ± 0.25 |
| 2 | 2 | tgtgcccagtCgTagccgaatagc | β-D-LNA, 11, 13 | 0.5 ± 0.3 |
| 3 | 3 | TgTgCcCaGtCgTaGcCgAaTaGc | β-D-LNA, 1, 3, 5, 7, 9, 11, 13, | <0.1 |
| | | | 15, 17, 19, 21, 23 | |
| 4 | 4 | tGtGcCcCgTcGtAgCcGaAtAgC | β-D-LNA, 2, 4, 6, 8, 10 12, 14, | <0.1 |
| | | | 16, 18, 20, 22, 24 | |
| 5 | 5 | tgtgcccagTcgtAgccgaatagc | β-D-LNA, 10, 14 | 5 ± 1 |
| 6 | 6 | tgtgcccagTcgtaGccgaatagc | β-D-LNA, 10, 15 | 5 ± 1 |
| 7 | 7 | tgtgcccagtCgTagccgaatagc | α-L-LNA, 11, 13 | 0.3 ± 0.2 |
| 8 | 8 | tgtgcccagTCgTagCCgaatagc | α-L-LNA, 10, 11, 13, 16, 17 | <0.1 |
| 9 | 9 | tgtgcccagTcgtAgccgaatagc | β-D-glycyl-amino LNA, 10,; β-D- | <0.1 |
| | | | LNA, 14 | |
| 10 | 10 | tgtgcccagTcgtAgccgaatagc | β-D-adamantyl-amino LNA, 10; β- | <0.1 |
| | | | D-LNA, 14 | |
| 11 | 11 | TGTGcccagtcgtagccgaATAGc | Methylphosphonate, 1, 2, 3, 4, | <0.1 |
| | | | 20, 21, 22, 23 | |
| 12 | 12 | TGTGCCCAGTCGTAGCCGAATAGC | Morpholino | <0.1 |
| 13 | 13 | tgtgCCCagtCgtagCCgaatagC | 5Me-dC, 5, 6, 7, 11, 17, 18, 24 | <0.1 |
| 14 | 14 | TGtgcccagtcgtagccgaataGC | 2′-O-Me inosine, 1, 2, 23, 24 | 1 ± 0.25 |
| 15 | 15 | TGtgcccagtCgTagccgaataGC | 2′-O-Me inosine, 1, 2, 11, 13, | <0.1 |
| | | | 23, 24 | |
| 16 | 16 | tgtgcccAgtcgtAgccgaatagc | 2-amino-adenine, 8, 14, | <0.1 |
| 17 | 17 | tgtgcccagTcgtAgccgaatagc | 6-chloro-2-methoxyacridine, 10, | <0.1 |
| | | | 14 | |
| 18 | 18 | TgtgcccagtcgtagccgaatagC | 6-chloro-2-methoxyacridine, 1, | <0.1 |
| | | | 24 | |
| 19 | 19 | tgtgcccagtCgUagccgaatagc | pdC, 11, pdU, 13 | 3 ± 1 |
| 20 | 20 | UgUgCCCagUCgUagCCgaaUagC | pdC, 5, 6, 7, 11, 16, 17, 24, | 6 ± 2 |
| | | | pdU, 1, 3, 10, 21 | |
| 21 | 21 | tgtgcccagTCgUAgccgaatagc | β-D-LNA, 10, 14; pdC, 11, pdU 13 | 9 ± 3 |
| 22 | 22 | UgUgCCCagTCgUAgCCgaaUagC | β-D-LNA, 10, 14; pdC, 5, 6, 7, | 13 ± 1 |
| | | | 11, 16, 17, 24, pdU, 1, 3, 10, | |
| | | | 21 | |

All oligonucleotides shared the same sequence and were designed to convert the stop codon (TAG) at Y22 of the kanamycin ORF into TAC (tyrosine) by TNE. The mismatch nucleotide is underlined. Lowercase letters represent unmodified DNA while uppercase letters represent modified nucleotides (base, sugar or phosphate backbone modifications or combinations thereof) and their position in the oligonucleotide. The modified nucleotides included in each oligonucleotide are stated. The TNE efficiency of each oligonucleotide is expressed as the fold increase (or decrease) of TNE compared with the TNE efficiency of the DNA only oligonucleotide (oligonucleotide 1). At least 4 replicates with each oligonucleotide were performed and each series of experiments also included multiple replicates of oligo 1 as a reference. To distinguish between different types of modified nucleotides located on the same oligonucleotide, one type of modified oligonucleotide is shown in bold (e.g oligonucleotides 9, 10, 21 & 22). pdC, 5-(1-propynyl)-2′-deoxycytidine; pdU, 5-(1-propynyl)-2′-deoxyuridine; LNA, locked nucleic acid; MP, methylphosphonate linkages; 5Me-dC, 5-methyl-deoxycytidine. In control experiments, no kanamycin resistant colonies were obtained when the oligonucleotide or protein was omitted from the reaction.

The oligonucleotides were designed to produce a single nucleotide substitution or insertion in the KmY22stop or KmY22Δ plasmids respectively, restoring the ORF function. The experiments demonstrated that the number and position of β-D-LNA nucleotides in the ss oligonucleotide is of relevance. An oligonucleotide (oligonucleotide 2) in which the β-D-LNA nucleotides are placed next to the mismatch nucleotide shows less TNE activity compared to the unmodified oligonucleotide. Increasing the number of β-D-LNA nucleotides (oligonucleotides 3 & 4) results in a biologically inactive oligonucleotide. However, an increase in TNE efficiency is observed when the β-D-LNA nucleotides are separated by 3 or 4 normal DNA nucleotides that include the mismatch nucleotide (oligonucleotides 5 & 6). Given the more DNA conformation adopted by α-L-LNA nucleotides, it may be expected that they would also enhance TNE to the same extent or perhaps even better than β-D-LNA. However, it was shown that oligonucleotides 7 & 8 are hardly active in our assay, demonstrating that the β-D-LNA stereoisomer is preferred for improvement in TNE. The 2′-amino-LNAs show superior DNA binding compared to other LNA forms due to the addition of extra groups to the LNA nucleotide that can provide additional DNA interactions (Singh et al. (1998) J. Org. Chem. 63, 10035). However, once again, although the binding affinity of oligonucleotides 9 & 10 is presumably enhanced, the 2′-amino-LNA derivatives tested eliminate the TNE activity of these oligonucleotides and hence are less preferred. Oligonucleotides 11, 12, 13 and 16, were not active in TNE in contrast to the strong enhancement they confer on antisense oligonucleotides. Oligonucleotide 14, containing 2 2′-O-Me inosine nucleotides at either end is as active as an unmodified oligonucleotide, but the oligonucleotide becomes inactive when the 2′-O-Me inosine nucleotides flank the mismatch nucleotide (oligonucleotide 15). The intercalator 6-chloro-2-methoxyacridine also renders the oligonucleotide inactive (oligonucleotide 17 & 18). Our data shows that oligonucleotides containing C5-propyne pyrimidines show an enhancement in TNE that is at least partially dependant upon the number of modified pyrimidines in the oligonucleotide. This result was surprising when we consider that an equivalent oligonucleotide containing C5-methyl cytosine (oligonucleotide 13) is inactive. Thus, the effect we observe is completely dependant upon the group attached to the C5 position of the pyrimidine. Finally, combination of β-D-LNA nucleotides with optimal spacing and C5-propyne pyrimidines on a single oligonucleotide shows an average of 13 fold enhancement in the TNE frequency above that of the unmodified oligonucleotide.

### Cell Free Experiments Using KmY22Δ

The optimal oligonucleotide designs were also tested for their ability to insert a single nucleotide and restore the functionality of the kanamycin ORF in plasmid KmY22Δ (see table 2). Using the unmodified DNA oligonucleotide, we found that its ability to introduce insertions via TNE was approximately 5 fold lower than its ability to produce substitutions. When we tested oligonucleotides 5, 19 and 20, containing LNA and/or C5-propyne pyrimidine nucleotides for their ability to introduce insertions, in contrast to our data on substitutions, these modified oligonucleotides did not enhance the efficiency above that obtained with the unmodified oligonucleotide (oligonucleotide 1). However, a synergistic increase in nucleotide insertion efficiency was obtained when oligonucleotides 21 or 22 were used. This demonstrated that both LNA and C5-propyne pyrimidines are preferably both present on the same oligonucleotide to enhance the efficiency of TNE whereby a nucleotide is inserted into the target. Furthermore, the difference in efficiency seen between oligonucleotide 20 and oligonucleotide 21 indicates that the number of C5-propyne pyrimidine nucleotides in the oligonucleotide may be maximized to obtain a optimal nucleotide insertion efficiency.

TABLE 2

Oligonucleotides containing LNA and/or C5-propyne pyrimidines were tested
in the cell free assay for their ability to perform a nucleotide insertion

| | | | | |
|---|---|---|---|---|
| SEQ ID # | Oligonucleotide | Sequence | Modification | TNE eff. |

| | | | | |
|---|---|---|---|---|
| 1 | 1 | tgtgcccagtcgtagccgaatagc | none | 1 ± 0.2 |
| 5 | 5 | tgtgcccagTcgtAgccgaatagc | β-D-LNA, 10, 14 | 1 ± 0.7 |
| 19 | 19 | tgtgcccagtCgUagccgaatagc | pdC, 11, pdU, 13 | 1.5 ± 0.5 |
| 20 | 20 | UgUgCCCagUCgUagCCgaaUagC | pdC, 5, 6, 7, 11, 16, 17, 24, | 1.5 ± 0.5 |
| | | | pdU, 1, 3, 10, 21 | |
| 21 | 21 | tgtgcccagTCgUAgccgaatagc | β-D-LNA, 10, 14; pdC, 11, pdU | 2.0 ± 0.75 |
| | | | 13 | |
| 22 | 22 | UgUgCCCagTCgUAgCCgaaUagC | β-D-LNA, 10, 14; pdC, 5, 6, 7, | 5.0 ± 1.0 |
| | | | 11, 16, 17, 24, pdU, 1, 3, | |
| | | | 10, 21 | |

### Sequence Analysis of Cell Free TNE Events

All the oligonucleotides in this study were designed to convert the stop codon (TAG) in KmY22stop to TAC, restoring the kanamycin ORF functionality. In order to establish the fidelity of repair of oligonucleotides containing modified nucleotides, plasmids were purified from kanamycin resistant colonies obtained after repair with either oligonucleotide 1 (unmodified DNA) or oligonucleotide 18 in which all the pyrimidine nucleotides were replaced by C5-propyne pyrimidines. When the TNE reaction was performed with oligonucleotide 1, all 40 plasmids sequenced showed the expected TAC repair event at Y22. However, when oligonucleotide 20 was used, 28/38 showed TAC at Y22 while the remaining 10 plasmids contained TAT at this position. Thus, while C5-propyne pyrimidines enhance the TNE frequency, they also influence the outcome of the repair reaction, making it more error prone

It was demonstrated that, using an in vitro TNE assay, the cell free system, oligonucleotides containing both C5-propyne pyrimidine nucleotides and a specific spacing of LNA's around the mismatch nucleotide do show significantly higher levels of TNE compared to the TNE efficiency obtained using normal DNA oligonucleotides. This enhancement can be as high as 14 fold. This enhancement could be improved further by targeting pyrimidine rich sequences so that percentage of C5-propyne pyrimidine nucleotides is maximised. Efficiency could also be further increased through the use of 7-propynyl purine nucleotides which enhance binding affinity to an even greater degree than C5-propyne pyrimidine nucleotides (He & Seela, 2002 Nucleic Acids Res. 30: 5485-5496). Combining propyne purines and pyrimidines allows the production of oligonucleotides in which all the nucleotides carry propynyl groups on the bases.

### Example 2

### TNE in Tobacco

### Tobacco Shoot Cultures

The source material for this example is tobacco in vitro shoot cultures, grown aseptically in glass jars (750 ml) in MS20 medium at a temperature of 25/20° C. (day/night) and a photon flux density of 80 μE·m⁻²·s⁻¹ (photoperiod of 16/24 h). MS20 medium is basic Murashige and Skoog's medium (Murashige, T. and Skoog, F., Physiologia Plantarum, 15: 473-497, 1962) containing 2% (w/v) sucrose, no added hormones and 0.8% Difco agar. The shoots are subcultured every 3 weeks to fresh medium.

### Protoplast Isolation

For the isolation of mesophyll protoplasts, fully expanded leaves of 3-6 week old shoot cultures are harvested. The leaves are sliced into 1 mm thin strips, which are then transferred to large (100 mm×100 mm) Petri dishes containing 45 ml MDE basal medium for a preplasmolysis treatment of 30 min. MDE basal medium contained 0.25 g KCl, 1.0 g MgSO₄.7H₂O, 0.136 g of KH₂PO₄, 2.5 g polyvinylpyrrolidone (MW 10,000), 6 mg naphthalene acetic acid and 2 mg 6-benzylaminopurine in a total volume of 900 ml. The osmolality of the solution is adjusted to 600 mOsm·kg⁻¹ with sorbitol, the pH to 5.7.

After preplasmolysis, 5 ml of enzyme stock is added to each Petri dish. The enzyme stock consists of 750 mg Cellulase Onozuka R10, 500 mg driselase and 250 mg macerozyme R10 per 100 ml, filtered over Whatman paper and filter-sterilized. The Petri dishes are sealed and incubated overnight in the dark at 25° C. without movement to digest the cell walls.

Next morning, the protoplast suspension is passed through 500 μm and 100 μm sieves into 250 ml Erlenmeyer flasks, mixed with an equal volume of KCl wash medium, and centrifuged in 50 ml tubes at 85×g for 10 min. KCl wash medium consisted of 2.0 g CaCl₂.2H₂O per liter and a sufficient quantity of KCl to bring the osmolality to 540 mOsm·kg⁻¹.

The centrifugation step is repeated twice, first with the protoplasts resuspended in MLm wash medium, which is the macro-nutrients of MS medium (Murashige, T. and Skoog, F., Physiologia Plantarum, 15: 473-497, 1962) at half the normal concentration, 2.2 g of CaCl₂.2H₂O per liter and a quantity of mannitol to bring the osmolality to 540 mOsm·kg⁻¹, and finally with the protoplasts resuspended in MLs medium, which is MLm medium with mannitol replaced by sucrose.

The protoplasts are recovered from the floating band in sucrose medium and resuspended in an equal volume of KCl wash medium. Their densities are counted using a haemocytometer. Subsequently, the protoplasts are centrifuged again in 10 ml glass tubes at 85×g for 5 min and the pellets resuspended at a density of 1×10⁵ protoplasts ml⁻¹ in electroporation medium. All solutions are kept sterile, and all manipulations are done under sterile conditions.

ALS Target Gene and Design of Oligonucleotides

In the tobacco acetolactate synthase (ALS) SurA gene (Gene Bank Accession X07644) the amino acid conversions P194Q and W571L render the ALS protein insensitive to the sulfonylurea herbicide chlorsulfuron. Two oligonucleotides have been designed to introduce a base pair mutation in the SurA codons coding for these amino acids. SEQ ID NO 23 will generate a P194Q mutation and SEQ ID NO 24 the W571L mutation. In both oligonucleotides all C and T residues are propynylated (in such an oligonucleotide deoxythymidine is replaced by 5-(1-propynyl)-2′-deoxyuridine), except for the deoxythymidine mismatch nucleotide in oligonucleotide SEQ ID NO 231, that corresponds to the position of the intended point mutation. Propynylated cytosine or uracil nucleotides are indicated Cp or Up in SEQ ID NO 23 and SEQ ID NO 24. Furthermore, at the positions two nucleotides away on either side from the mismatch nucleotide, LNA residues have been incorporated (indicated AA, T̂, Ĉ or Ĝ in SEQ ID NO 23 and SEQ ID NO 24). Control single strand oligonucleotides consist of only normal DNA residues (no C5 propyne pyrimidines nor LNA residues) with the same nucleotide sequences.

[SEQ ID NO: 23]

5′UpCpAGUpACpCpUpAUpCpAUpCpCpUpACpG\hat{ }UpTGC\hat{ }ACpUpUp
GACpCpUpGUpUpAUpAG3′

[SEQ ID NO: 24]

5′CpCpUpUpAUpAGAACpCpGAUpCpCpUpC\hat{ }CpAAT\hat{ }UpGAACpCp
ACpCpAUpUpCpCpCpAA3′

CodA Target Gene and Design of Oligonucleotides

For TNE aimed at base pair conversions, the ALS gene is a useful selectable marker gene, because single base changes at specific positions lead to single amino acid conversions, which in turn provide resistance to sulfonylurea herbicides. For TNE aimed at creating a single base insertion or deletion (indel), ALS is not useful as a selectable marker gene, because a single base indel will cause a frameshift of the Open Reading Frame of the gene, and thus a nonfunctional protein. Selection on sulfonylurea herbicides is therefore not possible.

CodA, a bacterial gene coding for cytosine deaminase, can be used as a negative selectable marker (Stougaard, Plant Journal 3: 755-761, 1993). Plant cells expressing this gene and exposed to 5-fluorocytosine (5-FC) will die from irreversible inhibition of the thymidylate synthase pathway by 5-fluorouracil (5-FU), which is formed by deamination of 5-FC catalyzed by the gene product of CodA. Frameshift mutations introduced in the CodA sequence by the action of oligonucleotides will render the gene nonfunctional, and such plant cells resistant to 5-FC.

In this example, the CodA negative selection system is exploited for the selection of tobacco cells undergoing an indel mutation due to the action of oligonucleotides. To this end, tobacco SR1 plants have been created that express the bacterial CodA gene from a CaMV 35S promoter, by Agrobacterium-mediated transformation as in Stougaard (Plant Journal 3: 755-761, 1993). Transformed plants have been tested for the correct response on 5-FC, and were maintained as in vitro shoot cultures as described above. In this example, a specific version of the CodA gene has been used, that has been optimized for plant codon usage in order to enhance its translation efficiency in plant cells. The sequence of the codon-optimized CodA gene is:

[SEQ ID NO: 25]
ATGTCTAACAACGCTCTTCAGACTATCATCAACGCTAGACTTCCTGGAGAAGAGGGAC
TTTGGCAGATTCATCTTCAGGATGGAAAGATCTCTGCTATCGATGCTCAGTCTGGAGTGATGC
CTATCACTGAGAACTCTCTTGATGCTGAGCAGGGACTTGTTATTCCTCCTTTCGTGGAGCCTC
ACATCCATCTTGATACAACTCAGACTGCTGGACAACCTAATTGGAACCAGTCTGGAACTCTTT
TCGAGGGAATCGAAAGATGGGCTGAGAGAAAGGCTCTTCTTACTCACGATGATGTGAAGCAAA
GGGCTTGGCAAACTCTTAAGTGGCAGATCGCTAACGGAATTCAGCATGTGAGGACTCATGTGG
ATGTGTCTGATGCTACTCTTACTGCTCTTAAGGCTATGCTGGAAGTGAAGCAGGAAGTCGCGC
CGTGGATTGATCTTCAGATCGTGGCTTTCCCTCAAGAGGGAATCCTTTCTTACCCTAACGGAG
AGGCTCTTCTTGAAGAGGCTCTTAGGCTTGGAGCTGATGTTGTTGGAGCTATCCCTCACTTCG
AGTTCACTAGGGAATACGGAGTTGAGTCTCTTCACAAGACTTTCGCTCTTGCTCAGAAGTACG
ATAGGCTTATCGATGTTCACTGCGATGAGATCGATGATGAGCAGTCAAGATTCGTTGAGACTG
TGGCTGCTCTTGCTCATCATGAAGGAATGGGAGCTAGAGTTACTGCTTCTCACACTACTGCTA
TGCACTCTTACAACGGAGCTTACACTTCTAGGCTTTTCGGCTTCTTAAGATGTCTGGTATCAA
CTTCGTGGCTAACCCTCTTGTGAACATCCATCTTCAGGGAAGATTCGATACTTACCCTAAGAG
GAGGGGAATTACTAGGGTGAAGGAGATGCTTGAGTCAGGTATCAATGTGTGCTTCGGACACGA
TGATGTTTTCGATCCTTGGTATCCTCTTGGAACTGCTAACATGCTTCAGGTGTTGCATATGGG
ACTTCATGTGTGCCAACTTATGGGATACGGACAGATCAACGATGGACTTAACCTTATCACTCA
CCACTCTGCTAGGACTCTTAACCTTCAGGATTACGGAATTGCTGCTGGAAACTCTGCTAACCT
TATCATCCTTCCTGCTGAGAATGGATTCGATGCTCTTAGGAGGCAAGTTCCTGTTAGGTACTC
TGTTAGGGGAGGAAAGGTGATCGCTTCTACTCAACCTGCTCAGACTACTGTTTACCTTGAGCA
GCCTGA

An oligonucleotide has been designed to introduce a single base pair insertion at a position in the 5′ end of the CodA gene. Propynylated cytosine or uracil nucleotides are indicated Cp or Up. At the positions two nucleotides away on either side from the nucleotide corresponding to the intended insertion (insertion nucleotide, underlined), LNA residues have been incorporated (indicated Â, V̂, Ĉ or Ĝ). Control single strand oligonucleotides consist of only normal DNA residues (no C5 propyne pyrimidines nor LNA residues) with the same nucleotide sequence.

[SEQ ID NO: 26]

5′GAAGUpCpUpAGCpGUpUpGAUpGA\hat{ }UpGAG\hat{ }UpCpUpGAAGAGCpGU
pUpGUpUpAGA3′

Protoplast Electroporation

Using PHBS as an electroporation medium (10 mM Hepes, pH 7.2; 0.2 M mannitol, 150 mM NaCl; 5 mM CaCl₂) and with a protoplast density in the electroporation mixture of ca. 1×10⁶ per ml, the electroporation settings are 250V (625 V cm⁻¹) charge and 800 μF capacitance with a recovery time between pulse and cultivation of 10 minutes. For each/electroporation ca. 1-2 μg oligonucleotide are used per 800 microliter electroporation=25 μg/ml.

Protoplast Regeneration and Selection

After the electroporation treatment, the protoplasts are placed on ice for 30 min to recover, then resuspended in T₀ culture medium at a density of 1×10⁵ protoplasts ml⁻¹. T₀ culture medium contained (per liter, pH 5.7) 950 mg KNO₃, 825 mg NH₄NO₃, 220 mg CaCl₂.2H₂O, 185 mg MgSO₄.7H₂O, 85 mg KH₂PO₄, 27.85 mg FeSO₄.7H₂O, 37.25 mg Na₂EDTA.2H₂O, the micro-nutrients according to Heller's medium (Heller, R., Ann Sci Nat Bot Biol Veg 14: 1-223, 1953), vitamins according to Morel and Wetmore's medium (Morel, G. and R. H. Wetmore, Amer. J. Bot. 38: 138-40, 1951), 2% (w/v) sucrose, 3 mg naphthalene acetic acid, 1 mg 6-benzylaminopurine and a quantity of mannitol to bring the osmolality to 540 mOsm·kg⁻¹.

The protoplasts resuspended in T₀ culture medium are then mixed with an equal volume of a solution of 1.6% SeaPlaque Low Melting Temperature Agarose in T₀ culture medium, kept liquid after autoclaving in a waterbath at 30° C. After mixing, the suspension is gently pipetted in 2.5 ml aliquots into 5 cm Petri dishes. The dishes are sealed and incubated at 25/20° C. (16/24 h photoperiod) in the dark.

After 8-10 days incubation in the dark, the agarose medium is cut into 6 equal pie-shaped parts, which are transferred to 10 cm Petri dishes each containing 22.5 ml of liquid MAP₁AO medium. This medium consisted of (per liter, pH 5.7) 950 mg KNO₃, 825 mg NH₄NO₃, 220 mg CaCl₂.2H₂O, 185 mg MgSO₄.7H₂O, 85 mg KH₂PO₄, 27.85 mg FeSO₄.7H₂O, 37.25 mg Na₂EDTA.2H₂O, the micro-nutrients according to Murashige and Skoog's medium (Murashige, T. and Skoog, F., Physiologia Plantarum, 15: 473-497, 1962) at one tenth of the original concentration, vitamins according to Morel and Wetmore's medium (Morel, G. and R. H. Wetmore, Amer. J. Bot. 38: 138-40, 1951), 6 mg pyruvate, 12 mg each of malic acid, fumaric acid and citric acid, 3% (w/v) sucrose, 6% (w/v) mannitol, 0.03 mg naphthalene acetic acid and 0.1 mg 6-benzylaminopurine. For purposes of selection of colonies with a successful TNE event, 41 nM chlorsulfuron, or 250 μg·ml⁻¹ 5-FC is also added to the medium. The Petri dishes are incubated at 25/20° C. in low light (photon flux density of 20 μE·m⁻²·s⁻¹) at a photoperiod of 16/24 h. After two weeks, the Petri dishes are transferred to full light (80 μE·m⁻²·s⁻¹). During this period of selection, most protoplasts died. Only protoplasts, in which through the action of the oligonucleotides a base change has occurred in the target gene so as to confer resistance to the herbicide or 5-FC, divide and proliferate into protoplast-derived microcolonies.

Six to eight weeks after isolation, the protoplast-derived colonies are transferred to MAP₁ medium. The agarose beads by this time fall apart sufficiently to transfer the microcolonies with a wide-mouthed sterile pipette, or else they are individually transferred with forceps. MAP₁ medium has the same composition as MAP₁AO medium, with however 3% (w/v) mannitol instead of 6%, and 46.2 mg·l⁻¹ histidine (pH 5.7). It was solidified with 0.8% (w/v) Difco agar.

After 2-3 weeks of growth on this solid medium, the colonies are transferred to regeneration medium RP, 50 colonies per 10 cm Petri dish. RP medium consisted of (per liter, pH 5.7) 273 mg KNO₃, 416 mg Ca(NO₃)₂.4H₂O, 392 mg Mg(NO₃)₂.6H₂O, 57 mg MgSO₄.7H₂O, 233 mg (NH₄)₂SO₄, 271 mg KH₂PO₄, 27.85 mg FeSO₄.7H₂O, 37.25 mg Na₂EDTA.2H₂O, the micro-nutrients according to Murashige and Skoog's medium (Murashige, T. and Skoog, F., Physiologia Plantarum, 15: 473-497, 1962) at one fifth of the published concentration, vitamins according to Morel and Wetmore's medium (Morel, G. and R. H. Wetmore, Amer. J. Bot. 38: 138-40, 1951), 0.05% (w/v) sucrose, 1.8% (w/v) mannitol, 0.25 mg zeatin and 41 nM chlorsulfuron, or 250 μg·ml⁻¹ 5-FC, and is solidified with 0.8% (w/v) Difco agar.

PCR Amplification of Target Gene

DNA is isolated from chlorsulfuron or 5-FC resistant resistant tobacco microcolonies using the DNeasy kit (Qiagen), and used as a template in a PCR reaction. Conversions of the targeted codons in the tobacco ALS gene are detected using the primers 5′GGTCAAGTGCCACGTAGGAT [SEQ ID NO: 27] & 5′GGGTGCTTCACTTTCTGCTC [SEQ ID NO: 28] that amplify a 776 bp fragment of this gene, including codon 194. The primers 5′CCCGTGGCAAGTACTTTGAT [SEQ ID NO:29] & 5′GGATTCCCCAGGTATGTGTG [SEQ ID NO:30] are likewise used to amplify 794 bps fragment of the tobacco ALS gene, including the codon 571. For PCR amplification of the modified CodA gene in 5-FC resistant tobacco calli, the following primer set was designed:

| | |
|---|---|
| 5′GTGGAAAAAGAAGACGTTCCAAC3′ | [SEQ ID NO: 31] |
| and | |
| 5′AGCATCGATAGCAGAGATCTTTC3′. | [SEQ ID NO: 32] |

Sequencing to Proof Nucleotide Conversion

Nucleotide conversion in the herbicide resistant tobacco callus is confirmed by sequencing the PCR products obtained from the DNA of such callus. Conversion of the tobacco ALS P194 codon (CCA to CAA) results in a double peak at the second position of the codon (C/A). Finally, conversion of the tobacco ALS W571 codon (TGG to TTG) results in a double peak at the second codon position (G/T).

### Example 3

### TNE in Mouse Embryonic Stem Cells

In order to have a selectable system to demonstrate TNE in mouse cells, a mouse embryonic stem (ES) cell line is used that expresses a selectable marker gene (neo) providing resistance to G418 in culture, but which has been rendered non-functional by a deliberate mutation. The objective of the TNE experiment is to repair this mutation and restore the functionality of the neo gene, resulting in selection of such cells in G418.

The mouse ES cell line is derived from line E14 (Te Riele et al., Proc. Natl. Acad. Sci. USA 89: 5128-5132, 1992) by introduction of a defective neo gene driven by the MC1 promoter. The defective neo gene contains two extra nucleotides GT immediately downstream from the ATG start codon of neo, resulting in a frameshift (Dekker et al., Nucl. Acids Res. 31, No. 6 e27, 2003). The cells are grown in BRL conditioned medium

For TNE experiments, cells were dispensed at a density of 7×10⁵ per well of a 6-well plate for 24 h. Then, to each well is added 1.4 ml serum-free medium, 10 μg of oligonucleotides and 63 μl of TransFast™ lipofection reagent (Promega). After 1 h, 4 ml of serum-containing medium is added, and the cells are incubated overnight. After 24 h of selection-free incubation, the cells are grown in selective medium containing 100 mg·l⁻¹ of G418. After 10 days, G418-resistant colonies are counted.

The oligonucleotide to be used to repair the frameshift mutation consists of a 36-mer corresponding to the sequence of the coding strand of the regio of the neo gene around the ATG start codon, but without the GT nucleotides that had been introduced in neo in order to create a frameshift mutation. Furthermore, all cytosine or thymidine nucleotides are propynylated, indicated below by Cp or Tp. At the positions two nucleotides away on either side from the frameshift insertion, LNA residues have been incorporated (indicated Â, T̂, Ĉ or Ĝ). Control single strand oligonucleotides consist of only normal DNA residues (no C5 propyne pyrimidines nor LNA residues), and contain the GT frameshift mutation. The oligonucleotide sequence is as follows:

[SEQ ID NO: 33]

5′TpCpTpAGAGCpCpGCpCpACpCpAT\hat{ }GAT\hat{ }CpACpCpGATp
GCpATpCpGAG3′

DNA is extracted from G418-resistant colonies, and subjected to PCR in order to amplify the region surrounding the neo start codon. The PCR fragments are subsequently sequenced in order to verify the correct repair of the frameshift mutation.

### Example 4

### Targeted Nucleotide Exchange in Tomato (Solanum Lycopersicum) Using C5-Propyne and LNA Modified Oligonucleotides

Acetolactate synthase (ALS, also referred to as acetohydroxy acid synthase; AHAS) is the first common enzyme in the biosynthetic pathway to the branched chain amino acids valine, leucine and isoleucine. The pathway exists in plants and microorganisms such as bacteria, fungi and algae. ALS is the primary target site of action for at least four structurally distinct classes of herbicides, including sulfonylureas (SU), imidazolinones (IMI), triazolopyrimidine sulfonamides (TP) and pyrimidinylsalicylates (PS). In tomato, ALS is a multicopy gene as two full length EST's are present in the Plant Transcript Database (http://planta.tigr.org). In our study we have defined transcript TA37274_{—}4081 as ALS1 and transcript TA37275_{—}4081 as ALS2. ALS1 encodes a protein of 659AA while ALS2 encodes a protein of 657AA. ALS1 and ALS2 show 93% and 96% identity at the DNA and protein levels respectively. The two proteins mainly differ in the signal peptide regions of the proteins responsible for chloroplast targeting. Despite these differences, both ALS1 and ALS2 proteins are both predicted to be targeted to the chloroplast. Previous studies have shown that several amino acid changes at conserved residues are sufficient to confer herbicide resistance to the plant, such as the P171Q, W548L & S627I mutations in rice ALS. The conservation of ALS between organisms is high and identical mutations in many organisms result in similar herbicide resistant phenotypes. In this study we have introduced normal DNA oligonucleotides or C5-propyne and LNA modified oligonucleotides into tomato leaf protoplasts designed to alter the P184 codon in ALS2 (or P184 in ALS1) to produce resistance to the sulfonylurea type herbicide chlorsulfuron. As the sequences of the modified and unmodified oligonucleotides are identical, any differences in the TNE efficiency must therefore be due to the C5-propyne and LNA modifications as we observed when using the cell free system.

### Materials and Methods

### Oligonucleotides

The sequences of the regions of ALS1 and ALS2 containing the P184/186 codons (uppercase) are shown below. Single nucleotide polymorphisms between ALS1 and ALS2 are shown in bold.

[SEQ ID NO 34]

ALS1 gattgttgctattacaggtcaagtgCCAaggaggatgattggtac
tgatgcgt P186

[SEQ ID NO 35]

ALS2 gattgttgctattaccggtcaagtgCCGaggaggatgattggtac
tgatgcgt P184

The oligonucleotides 44 and 95 in table 3 were designed to produce a P184Q alteration in ALS2. Both are “antisense” oligonucleotides, complementary to the non-transcribed strand of the ALS genes. Oligonucleotide 80 consists of repeats of GATC of C5-propyne and LNA modified nucleotides and serves as a control. The designs include phosphorothioate linkages between the terminal 4 nucleotides as such modifications are known to partially protect the oligonucleotide from degradation by nucleases.

TABLE 3

| SEQ ID NO | Oligo | Sequence | Mutation |
|---|---|---|---|
| SEQ ID NO 36 | 95 | A*T*C*A*TCCTCCTCTGCACT | P184Q |
| | | TG*A*C*C*G | |
| SEQ ID NO 37 | 44 | A*z*y*A*zyyzyyvyTGwAyz | P184Q |
| | | zG*A*y*y*G | |
| SEQ ID NO 38 | 80 | G*z*A*y*GzAyAxzCAxzAyG | None |
| | | zA*G*G*A*U | |

y=5-propynyl-2′-deoxycytidine; z=5-propynyl-2′-deoxyuracil; s=LNA A; v=LNA T; w=LNA C; x=LNA G; U=deoxyuracil; *=phosphorothioate linkages. All oligonucleotides were synthesized by Eurogentec and HPLC purified. The nucleotide that forms a mismatch with the target is underlined.

### Tomato Leaf Protoplast Isolation and Transformation

Isolation and regeneration of tomato leaf protoplasts has been previously described (Shahin, 1985 Theor. Appl. Genet. 69: 235-240; Tan et al. 1987 Theor. Appl. Genet. 75: 105-108; Tan et al. 1987 Plant Cell Rep. 6: 172-175) and the solutions required can be found in these publications. Briefly, Solanum lycopersicum seeds were sterilized with 0.1% hypochlorite grown in vitro on sterile MS20 medium in a photoperiod of 16/8 hours at 2000 lux at 25° C. and 50-70% relative humidity. 1 g of freshly harvested leaves were placed in a dish with 5 ml CPW9M and, using a scalpel blade, cut perpendicular to the main stem every mm. These were transferred a fresh plate of 25 ml enzyme solution (CPW9M containing 2% cellulose onozuka RS, 0.4% macerozyme onozuka R10, 2.4-D (2 mg/ml), NAA (2 mg/ml), BAP (2 mg/ml) pH5.8) and digestion proceeded overnight at 25° C. in the dark. The protoplasts were then freed by placing them on an orbital shaker (40-50 rpm) for 1 hour. Protoplasts were separated from cellular debris by passing them through a 50 μm sieve, and washing the sieve 2× with CPW9M. Protoplasts were centrifuged at 85 g, the supernatant discarded, and then taken up in half the volume of CPW9M. Protoplasts were finally taken up in 3 ml CPW9M and 3 ml CPW18S was then added carefully to avoid mixing the two solutions. The protoplasts were spun at 85 g for 10 mins and the viable protoplasts floating at the interphase layer were collected using a long pasteur pipette. The protoplast volume was increased to 10 ml by adding CPW9M and the number of recovered protoplasts was determined in a haemocytometer.

Oligonucleotides were introduced into tomato protoplasts by electroporation using a Gene Pulser (BioRad). The protoplasts were resuspended in PHBS as an electroporation medium (10 mM HEPES, pH 7.2; 0.2M mannitol, 150 mM NaCl, 5 mM CaCl₂) at a density of 1×10⁶/ml in 0.4 cm electroporation cuvettes. 5 μg of oligonucleotide was added to 1 ml of protoplast suspension, and electroporation was performed at 250V (625 C cm⁻¹) and 800 μF capacitance. Protoplasts were then carefully removed from the cuvette and transferred to a fresh tube and 8 ml of 9M medium was added. This was then spun at 85 g for 5 mins, the supernatant removed, and 2 ml of fresh 9M added.

Protoplasts were embedded in alginate solution for regeneration. 2 ml of alginate solution was added (mannitol 90 g/l, CaCl₂.2H₂O 140 mg/l, alginate-Na 20 g/l (Sigma A0602)) and was mixed thoroughly by inversion. 1 ml of this was layered evenly on a Ca-agar plate (72.5 g/1 mannitol, 7.35 g/l CaCl₂.2H₂O, 8 g/l agar) and allowed to polymerize. The alginate discs were then transferred to 4 cm Petri dishes containing 4 ml of K8p culture medium and incubated for 7 days in the dark at 30° C. Then discs were then cut up into 5 mm broad strips and layered on TM-DB callus induction medium containing 20 nM chlorsulfuron. Herbicide resistant calli appeared after 4-5 weeks incubation at 30° C., and individuals were then transferred to GM-ZG medium for further growth. After 2-3 weeks on this medium, part of the callus was used for DNA isolations.

### Analysis of Tomato Calli

Genomic DNA was isolated from 5 week old calli using the Plant DNAeasy Kit (Qiagen, #69104). Primers were designed to specifically amplify either ALS1 or ALS2. For ALS1 we used the primers 08Z769 (5′ GAAAGGGAAGGTGTTACGGATGTA SEQ ID NO 39) and 08Z770 (5′ CTTGATTGCGAACACCCACC SEQ ID NO 40); for ALS2 the primers 08Z773 (5′ GAAAGGGAAGGGGTTAAGGATGTG SEQ ID NO 41) and 08Z774 (5′ CTCGACTGTGAACACCCACC SEQ ID NO 42) were used. PCR amplification was performed using the proofreading enzyme rTth DNA polymerase XL (Roche) and the resulting PCR fragments were directly sequenced. To confirm the nucleotide changes, the blunt PCR fragments generated with the proofreading enzyme were A-tailed using Taq DNA polymerase (100 ng product (5 μl)+1 μl PCR buffer+1 μl dNTP's (20 mM)+1 U Taq DNA polymerase+2.8 μl H₂O; 10 mins at 72° C.). The PCR fragment was then purified using the PCR Purification Kit (Qiagen) and 10 ng was cloned into the TOPO TA cloning kit (Invitrogen). After transformation to E. coli, a PCR reaction was performed on white, kanamycin resistant colonies using rTth DNA polymerase XL (Roche) using M13 primers which anneal to sites in the vector and flank the insert. These PCR products were then directly sequenced using M13 primers.

### Results

Two independent experiments were performed to test whether the efficiency of TNE in tomato was enhanced using oligonucleotides containing C5-propyne and LNA modifications. The results are shown in table 4.

TABLE 4

| | | # | |
|---|---|---|---|
| | | chlorsulfuron | # calli per |
| | Protoplasts | resistant | 10⁶ |
| Oligonucleotide | transformed¹ | calli | protoplasts |
| 95 | 9 × 10⁶ | 1 | 0.11 |
| 44 | 4.5 × 10⁶ | 4 | 0.88 |
| 80 | 2.5 × 10⁶ | 0 | 0 |
| none | 9 × 10⁶ | 0 | 0 |

Oligonucleotide 44 containing both C5-propyne and LNA modified nucleotides gave approximately 8 fold more calli than oligo 95 consisting of unmodified DNA. The sequence of both of these oligonucleotides is identical. Oligonucleotide 80 is a “scrambled” oligonucleotide which also contains modified nucleotides. This oligonucleotide is not specific for either ALS1 or ALS2 and serves as a control to demonstrate that the presence of oligonucleotide alone in the plant cell is not sufficient to generate herbicide resistant calli. In addition, when oligonucleotide was omitted from the protoplast transformation mix, no herbicide resistant calli were observed, indicating that the level of spontaneous herbicide resistance in tomato protoplasts is below the detection level.

To demonstrate that the chlorsulfuron resistant calli did contain mutations at the expected site, 3 calli (1 from oligonucleotide 95 (D) and 2 from oligonucleotide 44 (E &F)) were sequenced. Sequence analysis of PCR products from ALS1 or ALS2 showed that in all 3 calli mixed peaks were present at the targeted codon. This demonstrates that the herbicide resistant phenotype was indeed due to nucleotide alterations produced by the oligonucleotides and that the calli were hemizygous for the nucleotide changes. We then proceeded to clone and sequence individual PCR products derived from ALS1 or ALS2. The results of the sequence analysis are shown in FIG. 5. Calli D and E showed a CCA to TCA change (P186S) at ALS2 and ALS1 respectively, while callus G showed a CCA to TTA alteration (P186L) at ALS1. Studies in tobacco have shown that any amino acid change at the conserved proline residue in the tobacco ALS orthologs (SurA or SurB) confer chlorsulfuron resistance (Kochevenko et al. 2003 Plant Phys. 132: 174-184). Therefore, the P186/184 codon of ALS1 or ALS2 is ideal to detect unexpected nucleotide changes, as such events will also lead to a herbicide resistant phenotype.

Our results demonstrate in general that the C5-propyne and LNA modifications do not inhibit the generation of nucleotide alterations in the genome and in particular in the genome of tomato. Furthermore, this type of modification increase the efficiency of targeted nucleotide exchange in plant cells in general significantly, in the case of tomato by approximately eight fold. This type of combined modification (LNA and propyne as described herein) provides a significant increase in vitro over earlier described cell free assays (i.e. in vitro) using only LNA modified oligonucleotides or only propyne modified nucleotides. Particular noteworthy here is the step from the cell free assay to an in vitro assay.

Surprisingly, we did not find the expected nucleotide change (CCG to CAG, P184Q, at ALS2). Instead, in calli D & E we observed a change (C to T) at the nucleotide 5′ to the targeted nucleotide in the codon. In addition, our data from callus G indicates that an oligonucleotide sharing a single mismatch with the target sequence is able to induce an alteration of 2 nucleotides. The sequence of the complete PCR products (500 bps) were compared with that of ALS1 or ALS2, and besides the changes at the targeted proline codon, no other nucleotide alterations were observed. Thus, an oligonucleotide is able to induce mutagenesis at a specific codon of a plant gene. Analysis of TNE events produced using the cell free system and a DNA oligonucleotide did indeed give the expected nucleotide change. In our analysis of callus D, also produced using an unmodified DNA oligonucleotide, we observed an unexpected nucleotide change. This suggests that there may be other factors in the cell which are affecting the repair process which do not play a role in the cell free system, making the step form a cell free assay to an in vivo assay not a simple one. However, we showed that an oligonucleotide containing C5-propyne modifications did produce unexpected nucleotide changes in the cell free system, suggesting that the modified nucleotides themselves are contributing to a more “error prone” repair. It is therefore noteworthy that callus G, showing a CCA to TTA change, was produced using a modified oligonucleotide.

Several studies have suggested that the nucleotide changes produced in human cells after treatment with oligonucleotides are due to integration of the oligonucleotide at the target site (Radecke et al. 2006 J. Gene Med. 8: 217-218). However, oligonucleotide integration cannot explain the results we have obtained in tomato. Firstly, oligonucleotide integration would result in the expected nucleotide alteration (P184Q) which we did not observe. Secondly, we also found nucleotide changes in ALS1 using oligonucleotides targeted to ALS2. Integration of this oligonucleotide at ALS1 would also change the single nucleotide polymorphism present in the third nucleotide of the P186/184 codon. As this was not observed, we conclude that oligonucleotide integration cannot explain our results. This may reflect a difference in the mechanism of TNE between animal and plant cells. We prefer mechanism in which the oligonucleotide binds to its genomic target and induces a mutagenic process at the targeted codon, whereupon the oligonucleotide is then degraded.

1.-41. (canceled)

42. An oligonucleotide for targeted alteration of a duplex DNA sequence, the duplex DNA sequence containing a first DNA sequence and a second DNA sequence which is the complement of the first DNA sequence, the oligonucleotide comprising a domain that is capable of hybridising to the first DNA sequence, which domain comprises at least one mismatch with respect to the first DNA sequence, and wherein the oligonucleotide comprises at least one section that contains at least two modified nucleotides having a higher binding affinity compared to naturally occurring A, C, T or G nucleotides, wherein
at least one modified nucleotide is a LNA that is positioned at a distance of at least one nucleotide from the at least one mismatch and wherein, optionally, the oligonucleotide contains at most about 50% LNA modified nucleotides; and
at least one modified nucleotide is a C7-propyne purine or a C5-propyne pyrimidine.

43. The oligonucleotide according to claim 42, wherein at least 2, 3, 4, 5 or 6 nucleotides are LNAs.

44. The oligonucleotide according to claim 42, wherein the LNAs are distributed independently over a distance of at most 10 nucleotides, and comprise at most 8, 6, 4, 3, or 2 nucleotides from both sides of the mismatch.

45. The oligonucleotide according to claim 42, wherein 2, 3, 4, 5 or 6 nucleotides are LNAs.

46. The oligonucleotide according to claim 42, wherein at most 10%, 20%, 25%, 30% or 40% of the modified nucleotides of the oligonucleotide are LNA derivatives.

47. The oligonucleotide according to claim 42, wherein the modified nucleotide is independently positioned on the 5′ side and/or on the 3′ side of the mismatch.

48. The oligonucleotide according to claim 42, wherein two LNA modified nucleotides located on one side of the 5′ or the 3′ side of the mismatch are separated from each other by at least one, or two nucleotides.

49. The oligonucleotide according to claim 42, wherein the purine is adenosine or guanosine and/or the pyrimidine is cytosine, uracil or thymidine.

50. The oligonucleotide according to claim 42, wherein, independently, at least 10% of the pyrimidines and/or purines are replaced by their respective propynylated derivatives, preferably at least 50%, more preferably at least 75% and most preferably at least 90%.

51. The oligonucleotide according to claim 42, wherein modified nucleotide is a pyrimidine.

52. The oligonucleotide according to claim 42, wherein modified nucleotide is a purine.

53. The oligonucleotide according to claim 42, wherein at least two modified nucleotides are propynylated nucleotides, independently selected from amongst propynylated purines and propynylated pyrimidines.

54. The oligonucleotide according to claim 42, wherein the oligonucleotide comprises at least 2 or 3 sections that independently contain at least 2, 3, 4, 5, 6, 7, 8, 9 or 10, modified nucleotides.

55. The oligonucleotide according to claim 42, wherein the sections are located near or at the 3′-end, the 5′-end and/or encompass or flank the position of the mismatch.

56. The oligonucleotide according to claim 42, wherein the nucleotide at the position of the mismatch is not modified.

57. The oligonucleotide according to claim 42, wherein at least one of the propyne modified nucleotides is located adjacent to the mismatch and within 2, 3, 4, 6, 7, 8, 9, or 10 nucleotides of the mismatch.

58. The oligonucleotide according to claim 42, having a length from 10 to 500 nucleotides.

59. The oligonucleotide according to claim 42, wherein the (modified) section is the domain.

60. A method for targeted alteration of a duplex acceptor DNA sequence, comprising combining the duplex acceptor DNA sequence with a donor oligonucleotide, wherein the duplex acceptor DNA sequence contains a first DNA sequence and a second DNA sequence which is the complement of the first DNA sequence and wherein the donor oligonucleotide comprises a domain that comprises at least one mismatch with respect to the duplex acceptor DNA sequence to be altered, preferably with respect to the first DNA sequence, and wherein the oligonucleotide comprises a section that contains at least one modified nucleotide having a higher binding affinity compared to naturally occurring A, C, T or G and wherein the modified nucleotide binds stronger to a nucleotide in an opposite position in the first DNA sequence as compared to a naturally occurring nucleotide complementary to the nucleotide in an opposite position in the first DNA sequence, in the presence of proteins that are capable of targeted nucleotide exchange and wherein the modified oligonucleotide is defined in claims 42.

61. The method according to claim 60, wherein the alteration is within a cell preferably selected from the group consisting of a plant cell, a fungal cell, a rodent cell, a primate cell, a human cell or a yeast cell.

62. The method according to claim 60, wherein the proteins are derived from a cell extract.

63. The method according to claim 60, wherein the cell extract is selected from the group consisting of a plant cell extract, a fungal cell extract, a rodent cell extract, a primate cell extract, a human cell extract or a yeast cell extract.

64. The method according to claim 60, wherein the alteration is a deletion, a substitution or an insertion of at least one nucleotide.

65. The method according to claim 60, wherein the cell is a eukaryotic cell, a plant cell, a non-human mammalian cell or a human cell.

66. The method according to claim 60, wherein the target DNA is from fungi, bacteria, plants, mammals or humans.

67. The method according to claim 60, wherein the duplex DNA is from genomic DNA, linear DNA, mammalian artificial chromosomes, bacterial artificial chromosomes, yeast artificial chromosomes, plant artificial chromosomes, nuclear chromosomal DNA, organelle chromosomal DNA, episomal DNA.

68. The method according to claim 60, for altering a cell, correcting a mutation by restoration to wild type, inducing a mutation, inactivating an enzyme by disruption of coding region, modifying bioactivity of an enzyme by altering coding region, modifying a protein by disrupting the coding region.

69. The method for altering a cell, correcting a mutation by restoration to wild type, inducing a mutation, inactivating an enzyme by disruption of coding region, modifying bioactivity of an enzyme by altering coding region, modifying a protein by disrupting the coding region, mismatch repair, targeted alteration of (plant) genetic material, including gene mutation, targeted gene repair and gene knockout using an oligonucleotide as defined in claim 42.

70. The method for enhanced targeted alteration of a duplex DNA sequence, the duplex DNA sequence containing a first DNA sequence and a second DNA sequence which is the complement of the first DNA sequence, using an oligonucleotide as defined in claim 42;
the oligonucleotide comprising a domain that is capable of hybridising to the first DNA sequence, which domain comprises at least one mismatch with respect to the first DNA sequence, and wherein the oligonucleotide comprises a section that contains at least two modified nucleotides having a higher binding affinity compared to naturally occurring A, C, T or G nucleotides, wherein
at least one modified nucleotide is a LNA that is positioned at a distance of at least one nucleotide from the at least one mismatch and wherein, optionally, the oligonucleotide contains at most about 50% LNA modified nucleotides; and
at least one modified nucleotide is a C7-propyne purine or a C5-propyne pyrimidine;

71. A method for providing herbicide resistance to plants using an oligonucleotide as defined in claim 42.

72. A kit comprising an oligonucleotide as defined in claim 42.

73. A modified genetic material produced by the method of claim 60.

74. A cell comprising the modified genetic material of claim 73.

75. A method for increasing targeted nucleotide exchange efficiency of a duplex DNA, comprising
(a) obtaining an oligonucleotide comprising a domain that is capable of hybridizing to a first DNA sequence of said duplex, wherein said domain comprises:
(i) at least one mismatch with respect to the first DNA sequence; and
(ii) at least one modified nucleotide having an increased binding affinity, and
(b) decreasing the distance between said modified nucleotide and said mismatch to about 8 or fewer nucleotides; and
(c) recovering a oligonucleotide for use in targeted nucleotide exchange.

76. An oligonucleotide for targeted alteration of a duplex DNA, wherein said oligonucleotide comprises a domain that is capable of hybridizing to a first DNA sequence of said duplex and said domain comprises:
(a) at least one mismatch with respect to the first DNA sequence;
(b) at least one section comprising at least one modified nucleotide having an increased binding affinity, wherein said modified nucleotide is a LNA,
wherein said modified nucleotide is positioned at most 8 nucleotides from said mismatch.

77. The oligonucleotide of claim 76, wherein modified nucleotide is positioned at most 8 nucleotides from said mismatch.

78. The oligonucleotide of claim 76, wherein modified nucleotide is positioned at most 6 nucleotides from said mismatch.

79. The oligonucleotide of claim 76, wherein modified nucleotide is positioned at most 4 nucleotides from said mismatch.

80. The oligonucleotide of claim 76, wherein modified nucleotide is positioned at most 2 nucleotides from said mismatch.

81. The oligonucleotide of claim 76, wherein said domain comprises 2 LNAs.

82. An oligonucleotide for targeted nucleotide exchange of a duplex DNA, wherein said oligonucleotide comprises
(a) a modified nucleotide; and
(b) a mismatch with respect to a strand of said duplex DNA,
wherein said modified nucleotide is positioned about 1 nucleotide away from said mismatch.


SEQUENCE LISTING (42 sequences)

SEQ ID NO: 1 - DNA, 24 bp, Artificial
  non-modified sequence for TNE
tgtgcccagtcgtagccgaatagc

SEQ ID NO: 2 - DNA, 24 bp, Artificial
  probe for TNE
tgtgcccagtcgtagccgaatagc

SEQ ID NO: 3 - DNA, 24 bp, Artificial
  probe for TNE
tgtgcccagtcgtagccgaatagc

SEQ ID NO: 4 - DNA, 24 bp, Artificial
  probe for TNE
tgtgccccgtcgtagccgaatagc

SEQ ID NO: 5 - DNA, 24 bp, Artificial
  probe for TNE
tgtgcccagtcgtagccgaatagc

SEQ ID NO: 6 - DNA, 24 bp, Artificial
  probe for TNE
tgtgcccagtcgtagccgaatagc

SEQ ID NO: 7 - DNA, 24 bp, Artificial
  Probe for TNE
tgtgcccagtcgtagccgaatagc

SEQ ID NO: 8 - DNA, 24 bp, Artificial
  Probe for TNE
tgtgcccagtcgtagccgaatagc

SEQ ID NO: 9 - DNA, 24 bp, Artificial
  Probe for TNE
tgtgcccagtcgtagccgaatagc

SEQ ID NO: 10 - DNA, 24 bp, Artificial
  Probe for TNE
tgtgcccagtcgtagccgaatagc

SEQ ID NO: 11 - DNA, 24 bp, Artificial
  Probe for TNE
tgtgcccagtcgtagccgaatagc

SEQ ID NO: 12 - DNA, 24 bp, Artificial
  Probe for TNE
tgtgcccagtcgtagccgaatagc

SEQ ID NO: 13 - DNA, 24 bp, Artificial
  Probe for TNE
tgtgcccagtcgtagccgaatagc

SEQ ID NO: 14 - DNA, 24 bp, Artificial
  Probe for TNE
tgtgcccagtcgtagccgaatagc

SEQ ID NO: 15 - DNA, 24 bp, Artificial
  Probe for TNE
tgtgcccagtcgtagccgaatagc

SEQ ID NO: 16 - DNA, 24 bp, Artificial
  Probe for TNE
tgtgcccagtcgtagccgaatagc

SEQ ID NO: 17 - DNA, 24 bp, Artificial
  Probe for TNE
tgtgcccagtcgtagccgaatagc

SEQ ID NO: 18 - DNA, 24 bp, Artificial
  Probe for TNE
tgtgcccagtcgtagccgaatagc

SEQ ID NO: 19 - DNA, 24 bp, Artificial
  Probe for TNE
tgtgcccagtcguagccgaatagc

SEQ ID NO: 20 - DNA, 24 bp, Artificial
  probe for TNE
ugugcccagucguagccgaauagc

SEQ ID NO: 21 - DNA, 24 bp, Artificial
  probe for TNE
tgtgcccagtcguagccgaatagc

SEQ ID NO: 22 - DNA, 24 bp, Artificial
  probe for TNE
ugugcccagtcguagccgaauagc

SEQ ID NO: 23 - DNA, 40 bp, Artificial
  probe for TNE
ucaguaccuaucauccuacgutgcacuugaccuguuauag

SEQ ID NO: 24 - DNA, 40 bp, Nicotiana benthamiana
  modified_base (1)..(2): 5-(1-propynyl)-2′-deoxycytidine
ccuuauagaaccgauccuccaatugaaccaccauucccaa

SEQ ID NO: 25 - DNA, 1261 bp, Nicotiana benthamiana
atgtctaacaacgctcttcagactatcatcaacgctagacttcctggagaagagggactttggcagattcatcttcaggatggaaagatctctgctatcgatgctcagtctggagtgatgcctatcactgagaactctcttgatgctgagcagggacttgttattcctcctttcgtggagcctcacatccatcttgatacaactcagactgctggacaacctaattggaaccagtctggaactcttttcgagggaatcgaaagatgggctgagagaaaggctcttcttactcacgatgatgtgaagcaaagggcttggcaaactcttaagtggcagatcgctaacggaattcagcatgtgaggactcatgtggatgtgtctgatgctactcttactgctcttaaggctatgctggaagtgaagcaggaagtcgcgccgtggattgatcttcagatcgtggctttccctcaagagggaatcctttcttaccctaacggagaggctcttcttgaagaggctcttaggcttggagctgatgttgttggagctatccctcacttcgagttcactagggaatacggagttgagtctcttcacaagactttcgctcttgctcagaagtacgataggcttatcgatgttcactgcgatgagatcgatgatgagcagtcaagattcgttgagactgtggctgctcttgctcatcatgaaggaatgggagctagagttactgcttctcacactactgctatgcactcttacaacggagcttacacttctaggcttttcggcttcttaagatgtctggtatcaacttcgtggctaaccctcttgtgaacatccatcttcagggaagattcgatacttaccctaagaggaggggaattactagggtgaaggagatgcttgagtcaggtatcaatgtgtgcttcggacacgatgatgttttcgatccttggtatcctcttggaactgctaacatgcttcaggtgttgcatatgggacttcatgtgtgccaacttatgggatacggacagatcaacgatggacttaaccttatcactcaccactctgctaggactcttaaccttcaggattacggaattgctgctggaaactctgctaaccttatcatccttcctgctgagaatggattcgatgctcttaggaggcaagttcctgttaggtactctgttaggggaggaaaggtgatcgcttctactcaacctgctcagactactgtttaccttgagcagcctga

SEQ ID NO: 26 - DNA, 41 bp, Artificial
  probe for tne
gaagucuagcguugaugaugagucugaagagcguuguuaga

SEQ ID NO: 27 - DNA, 20 bp, Artificial
  primer
ggtcaagtgccacgtaggat

SEQ ID NO: 28 - DNA, 20 bp, Artificial
  primer
gggtgcttcactttctgctc

SEQ ID NO: 29 - DNA, 20 bp, Artificial
  primer
cccgtggcaagtactttgat

SEQ ID NO: 30 - DNA, 20 bp, Artificial
  primer
ggattccccaggtatgtgtg

SEQ ID NO: 31 - DNA, 23 bp, Artificial
  primer
gtggaaaaagaagacgttccaac

SEQ ID NO: 32 - DNA, 23 bp, Artificial
  primer
agcatcgatagcagagatctttc

SEQ ID NO: 33 - DNA, 35 bp, Artificial
  probe for tne
tctagagccgccaccatgatcaccgatgcatcgag

SEQ ID NO: 34 - DNA, 53 bp, Lycopersicon esculentum
gattgttgctattacaggtcaagtgccaaggaggatgattggtactgatgcgt

SEQ ID NO: 35 - DNA, 53 bp, Lycopersicon esculentum
gattgttgctattaccggtcaagtgccgaggaggatgattggtactgatgcgt

SEQ ID NO: 36 - DNA, 24 bp, Artificial
  probe for TNE
atcatcctcctctgcacttgaccg

SEQ ID NO: 37 - DNA, 24 bp, artificial
  probe for tne
atcatcctcctctgcacttgaccg

SEQ ID NO: 38 - DNA, 24 bp, artificial
  probe for tne
gtacgtacagtcagtacgtaggat

SEQ ID NO: 39 - DNA, 24 bp, artificial
  primer
gaaagggaaggtgttacggatgta

SEQ ID NO: 40 - DNA, 20 bp, artificial
  primer
cttgattgcgaacacccacc

SEQ ID NO: 41 - DNA, 24 bp, artificial
  primer
gaaagggaaggggttaaggatgtg

SEQ ID NO: 42 - DNA, 20 bp, artificial
  primer
ctcgactgtgaacacccacc